SEQUENCE LISTING (22 sequences)

SEQ ID NO: 1 - protein, 6 aa, Artificial Sequence
  Synthetic
HSSKLQ

SEQ ID NO: 2 - protein, 7 aa, Artificial Sequence
  Synthetic
KGISSQY

SEQ ID NO: 3 - protein, 7 aa, Artificial Sequence
  Synthetic
SRKSQQY

SEQ ID NO: 4 - protein, 7 aa, Artificial Sequence
  Synthetic
GQKGQHY

SEQ ID NO: 5 - protein, 7 aa, Artificial Sequence
  Synthetic
EHSSKLQ

SEQ ID NO: 6 - protein, 7 aa, Artificial Sequence
  Synthetic
QNKISYQ

SEQ ID NO: 7 - protein, 7 aa, Artificial Sequence
  Synthetic
ENKISYQ

SEQ ID NO: 8 - protein, 7 aa, Artificial Sequence
  Synthetic
ATKSKQH

SEQ ID NO: 9 - protein, 7 aa, Artificial Sequence
  Synthetic
KGLSSQC

SEQ ID NO: 10 - protein, 7 aa, Artificial Sequence
  Synthetic
LGGSQQL

SEQ ID NO: 11 - protein, 7 aa, Artificial Sequence
  Synthetic
QNKGHYQ

SEQ ID NO: 12 - protein, 7 aa, Artificial Sequence
  Synthetic
TEERQLH

SEQ ID NO: 13 - protein, 7 aa, Artificial Sequence
  Synthetic
GSFSIQH

SEQ ID NO: 14 - protein, 8 aa, Artificial Sequence
  Synthetic
CHSSLKQK

SEQ ID NO: 15 - protein, 15 aa, Artificial Sequence
  Synthetic
CEEEEHSSLKQKKKK

SEQ ID NO: 16 - protein, 7 aa, Artificial Sequence
  Synthetic
KGISSQY

SEQ ID NO: 17 - protein, 6 aa, Artificial Sequence
  Synthetic
HSSKLQ

SEQ ID NO: 18 - protein, 6 aa, Artificial Sequence
  Synthetic
HSSKLQ

SEQ ID NO: 19 - protein, 6 aa, Artificial Sequence
  Synthetic
HSSKXQ

SEQ ID NO: 20 - protein, 6 aa, Artificial Sequence
  Synthetic
HSSKXQ

SEQ ID NO: 21 - protein, 6 aa, Artificial Sequence
  Synthetic peptide
HSSKXQ

SEQ ID NO: 22 - protein, 6 aa, Artificial Sequence
  Synthetic peptide
HSSKXQ

(19) United States
(12) Patent Application Publication
(10) Pub. No.: US 2014/0134658 A1
(43) Pub. Date: May 15, 2014

(54) PSA ENZYMATIC ACTIVITY: A NEW BIOMARKER FOR ASSESSING PROSTATE CANCER AGGRESSIVENESS

(71) Applicants: NORTHWESTERN UNIVERSITY, AN ILLINOIS NOT FOR PROFIT CORPORATION, Evanston, IL (US); OHMX CORPORATION, Evanston, IL (US)

(72) Inventors: Michael J. Ahrens, Evanston, IL (US); Byron Anderson, Morton Grove, IL (US); Paul A. Bertin, Chicago, IL (US); William J. Catalona, Chicago, IL (US); Dimitra Georganopoulou, Chicago, IL (US)

(73) Assignees: NOTRHWESTERN UNIVERSITY, AN ILLINOIS NOT FOR PROFIT CORPORATION, Evanston, IL (US); OHMX CORPORATION, Evanston, IL (US)

(21) Appl. No.: 14/033,169

(22) Filed: Sep. 20, 2013

Related U.S. Application Data
(63) Continuation-in-part of application No. 13/068,938, filed on May 23, 2011.
(60) Provisional application No. 61/475,496, filed on Apr. 14, 2011.
(60) Provisional application No. 61/437,056, filed on Jan. 28, 2011.
(60) Provisional application No. 61/394,458, filed on Oct. 19, 2010.
(60) Provisional application No. 61/347,121, filed on May 21, 2010.
(60) Provisional application No. 61/709,680, filed on Oct. 4, 2012.

(51) Int. Cl. C12Q 1/37

(52) U.S. Cl. CPC C12Q 1/37
USPC 435/23

(57) ABSTRACT

The disclosure provides for methods to determine prognosis, aggressiveness, and progression of prostate cancer with the ability to differentiate between aggressive and non-aggressive prostate cancer. The method utilizes the differential enzymatic activity in patient samples, for example of enzymatically active prostate specific antigen (PSA) activity, in order to determine the aggressiveness and prognosis of prostate cancer, and monitor the progression of prostate cancer therapy. The invention also encompasses assay platforms (e.g., optical or electrochemical) that specifically detect PSA-triggered peptide cleavage events.

### CROSS REFERENCED AND RELATED PATENT APPLICATIONS

This application is continuation-in-part of U.S. patent application Ser. No. 13/068,938, filed on May 23, 2011, which claims priority under 35 U.S.C. §119(e) to U.S. provisional application 61/347,121, filed May 21, 2010, U.S. provisional application 61/394,458, filed Oct. 19, 2010, U.S. Provisional application 61/437,056, filed Jan. 28, 2011, and U.S. provisional application 61/475,496, filed Apr. 14, 2011, and also claims priority to U.S. Provisional application 61/709,680, filed Oct. 4, 2012, all hereby incorporated by reference in their entirety, and in particular for their figures.

This application is related to International application No. PCT/US11/00919, filed May 23-2011 (published as WO 2011/146143A3, Nov. 24, 2011), and U.S. Provisional application No. 61/347,121 filed May 21, 2010, both of which are incorporated by reference in their entirety.

### BRIEF SUMMARY

Prostate carcinoma is the most common type of cancer in men. Over 200,000 new cases are identified each year and over 30,000 will die from this disease this year alone. Detection of prostate cancer early provides the best opportunity for a cure. Although prostate specific antigen (PSA) is considered as an effective tumor marker, it is not cancer specific. There is considerable overlap in PSA concentrations in men with prostate cancer and men with benign prostatic diseases. Furthermore, PSA levels cannot be used to differentiate men with indolent or organ confined prostate cancer (who would benefit from surgery) from those men with aggressive or non-organ confined prostate cancer (who would not benefit from surgery).

At present, serum PSA measurement, in combination with digital rectal examination (DRE), represents the leading tool used to detect and diagnose prostate cancer.

Commercially-available PSA assays are commonly performed in regional or local laboratories. These assays play a part in the current strategy for early detection of prostate cancer. A problem arises, however, when a modestly abnormal PSA value (4-10 ng/ml) is encountered in the context of a negative DRE. Only 20-30% of individuals with such findings will demonstrate carcinoma on biopsy. Kantoff and Talcott, 8(3) Hematol. Oncol. Clinics N Amer 555 (1994)).

In addition, PSA is not a disease-specific marker, as elevated levels of PSA are detectable in a large percentage of patients with benign prostatic hyperplasia (BPH) and prostatitis (25-86%) (Gao et al., 1997, Prostate 31: 264-281), as well as in other nonmalignant disorders, which significantly limits the diagnostic specificity of this marker. For example, elevations in serum PSA of between 4 to 10 ng/ml are observed in BPH, and even higher values are observed in prostatitis, particularly acute prostatitis.

BPH is an extremely common condition in men. Further confusing the situation is the fact that serum PSA elevations may be observed without any indication of disease from DRE, and vice-versa. Moreover, it is now recognized that PSA is not prostate-specific (Gao et al., for review). Despite original assumptions that PSA was a tissue-specific and gender-specific antigen, immunohistochemical and immunoassay methods have detected PSA in female and male periurethral glands, anal glands, apocrine sweat glands, apocrine breast cancers, salivary gland neoplasms, and most recently in human breast milk.

Cancer of the prostate is the second most common cause of cancer-related mortality among men. Hahnfeld L E and Moon T D (1999) Medical Clinical North America, 83(5), 1231-45. Because advanced disease is incurable, efforts have focused on identifying prostate cancer at an early stage, when it is confined to the prostate and therefore more amenable to cure. Unfortunately, prostate cancer can remain asymptomatic until tumor metastasis affects other organs or structures.

Screening for prostate cancer is primarily done by the detection of PSA in the blood. The diagnostic value of PSA for prostate cancer is limited, due to its lack of specificity between benign and cancerous conditions. Egawa et al., (1999) Int. J. Urology, 6, 493-501. As a result, benign conditions such as benign prostatic hyperplasia (BPH), prostatitis and infarction, as well as prostatic intraepithelial neoplasia, can be associated with elevated serum levels of PSA. In addition to PSA serum levels, other diagnostic methods are used, including digital rectal examination (DRE) and transrectal ultrasonography (TRUS).

In fact, approximately two thirds of all elevated PSA levels (>4 ng/ml) in men over the age of 50 are due to BPH or prostatitis. Stenman et al. (1999) Cancer Biology, 9, 83-93. Thus, merely establishing that a patient has elevated levels of PSA is not diagnostic of cancer, and further tests are necessary. Because of this lack of specificity more than one million men with elevated PSA levels undergo prostate biopsy; yet, only 1 of 4 are diagnosed with cancer.

Moreover, among those patients identified with prostate cancer, current PSA screening methods are unable to differentiate between aggressive disease, warranting radical treatment, and indolent disease, where “watchful waiting” is preferable.

Since the early identification of PSA and proposal to use serum PSA as a first-line screening tool, an abundance of reports have emerged attempting to improve the sensitivity, specificity, and prognostic ability to discern the aggressiveness of prostate cancer. Within these reports, many have proposed the use of isoforms of PSA, PSA in relation to prostate volume (density), change in PSA over time (PSA velocity), and metabolomic profiles as potential adjunct biomarkers to aid in predicting the development and progression of the disease. Common to all of these reports is the observation that each biomarker is positively associated with cancer staging. Therefore, a cutoff value for treatment versus no treatment cannot be established, as all the non-aggressive cases are also found within range of the aggressive cases.

A need therefore exists for an assay which can specifically identify prostate cancer, can distinguish prostate cancer from benign hyperplasia, can identify prostate cancer even though PSA levels are low, and identify the aggressiveness of cancer and stages of disease progression. Therefore, it is important to develop strategies that increase the positive predictive value of PSA testing. The proteolytic activity of PSA was identified in the early 1980's (Ban, Y. et al (1984) Biochem Bioph Res Co 123, 482-488), followed by its natural substrate identification and classification as a serine protease from the kallikrein family. Lilja, H. (1985) Journal of Clinical Investigation 76, 1899-1903. Synthetic peptide substrates developed as pro-drug components were later reported and shown to be specific to PSA with undetectable cross-reactivity against a panel of extracellular proteases. Denmeade, S. (1997) Cancer Research 57, 4924-4930. Tagging these peptides with fluorophores proximal to the PSA proteolytic site enabled spectroscopic detection of PSA enzymatic activity.

### BRIEF DESCRIPTION OF THE DRAWINGS

FIG. 1 shows the structures of several prostate cancer specific peptides (PCSPs), including mor-HSSKLQ-AMC (sometimes referred to herein as “AMIDE”), Mor-HSSK-Hiv-Q-AMC (sometimes referred to herein as “HIV”, and Mor-HSSK-Hic-Q-AMC (sometimes referred to herein as “HIC”).

FIGS. 2A and 2B depict the following 2A: Box plot for Prostate Specific Antigen Proteolytic Activity (PPA) for distributed populations of aggressive and non-aggressive prostate cancer. 2B: ROC curve analyses. Enzymatic activity level of PSA (AUC=0.7008); ratio of enzymatic activity level of PSA to serum PSA, i.e. rPSA (AUC=0.7784).

FIGS. 3A, 3B, 3C, and 3D depict the following 3A Fluorescence time course for a serial dilution of a genuine, purified sample of PSA from 2615 to 26 ng/mL. 3B Standard curve generated from a serial dilution of PSA 3C Fluorescence time course for 38 prostatic fluid samples, 3D Correlated enzymatic activity levels of PSA for different prostatic fluid samples for patients all diagnosed with aggressive or non-aggressive cancer. The shaded box indicates the number of diagnosed patients that would truly qualify for active surveillance and as a result avoid unnecessary surgery or other clinical intervention.

FIG. 4 depicts the scatter graph for the amount/levels of total PSA present in prostatic fluids with great overlap between aggressive and non-aggressive cancer patients.

FIG. 5 shows the scatter graph for the enzymatic activity level of PSA for 20 EPS samples as they predict upstaging and upgrading between clinically defined and surgically defined values for patients that are diagnosed with prostate cancer, and their clinical levels are compared to their pathology levels. The shaded box again indicates the number of diagnosed patients that would truly qualify for active surveillance, based on the pathology levels and as a result avoid unnecessary surgery or other clinical intervention.

FIGS. 6A, 6B and 6C shows average measurements of PSA activity in serum or post-DRE urine for the purpose of diagnosing cancer, i.e. differentiating between benign conditions (like Benign Prostatic Hyperplasia, i.e. BPH) and cancer. FIG. 6A shows the lack of correlation between the serum total PSA levels (Serum tPSA) values of patients and the presence of cancer. FIG. 6B shows the detection of enzymatic activity against the HSSKLQ peptide present in “post prostatic massage urine” (post digital rectal examination prostatic massage) of patients with prostate cancer relative to those with benign disease. In this assay 30 samples were screened for enzymatic activity. The samples included 15 biopsy confirmed prostate cancer patients with Gleason scores of 6 or greater and 15 samples from patients with normal prostate biopsies but diagnosed with BPH. Enzymatic activity against the HSSKLQ peptide was assayed as described in Downes et al. (2006) B. J. U. International 99:263-268. As depicted, the majority of samples from patients with benign disease showed minimal cleavage of the HSSKLQ peptide, in contrast to the relatively high median activity witnessed in samples from patients with biopsy-confirmed prostate cancer. FIG. 6C shows that the normalization of enzymatic activity on the basis of prostate volume provides improved correlation between enzymatic activity in post prostatic massage urine of patients with prostate cancer relative to those with benign disease.

FIGS. 7A, 7B, 7C and 7D depict receiver operator characteristic (ROC) curves used for diagnosis of prostate cancer, in particular for 7A total prostate specific antigen (t-PSA) using a commercially approved test (area under the curve 0.50), 7B enzymatic activity against the HSSKLQ peptide in post prostatic massage urine (area under the curve 0.58), 7C enzymatic activity against HSSKLQ normalized for total PSA in post massage urine (area under the curve 0.64) and 7D enzymatic activity against HSSKLQ normalized for prostate volume (area under the curve 0.74).

FIGS. 8A, 8B, 8C and 8D depict receiver operator characteristic (ROC) curves for diagnosing Prostate cancer, in a population of patients with BPH and patients with cancer obtained in the follow on study. 8A Total prostate specific antigen (t-PSA) using a commercially approved test (area under the curve 0.34), 8B enzymatic activity against the HSSKLQ peptide in post prostatic massage urine (area under the curve 0.47), 8C enzymatic activity against HSSKLQ normalized for total PSA in post massage urine (area under the curve 0.54) and 8D enzymatic activity against HSSKLQ normalized for prostate volume (area under the curve 0.51).

FIGS. 9A, 9B and 9C depict a follow on study wherein the enzymatic activity against the HSSKLQ peptide present in post prostatic massage urine was assayed in a further 47 samples shown here in log values, for the purposes of diagnosis prostate cancer. In this assay, urine auto-fluorescence was subtracted from the fluorescence due to enzymatic activity observed in the optical assay. 9A Serum t-PSA levels measured by commercially approved PSA assay in patients with benign disease and those with prostate cancer and 9B measurement of enzymatic activity against HSSKLQ in these same patient samples. Unexpectedly, the serum t-PSA value actually appeared to function as a negative biomarker for prostate cancer; that is, the observed mean for cancer patients was higher than the mean of those with benign prostatic hyperplasia. However, as observed in the earlier study, the mean of enzymatic activity remained higher in prostate cancer patients relative to those with benign disease. 9C depicts results from the follow on study in which the enzymatic activity on the basis of prostate volume again showed improved discrimination between patients with prostate cancer relative to those with benign disease.

### BRIEF SUMMARY OF THE INVENTION

In some embodiments, a method of diagnosing prostate cancer in a subject is provided, the disclosed method encompassing determining the level of enzymatic activity, for example, proteolytic activity, in a sample from the subject wherein the sample is, for example, urine, semen, prostatic fluid or post prostatic massage urine; and correlating the level of enzymatic activity to the presence of prostate cancer.

In some embodiments, the method of diagnosing prostate cancer in a subject encompasses determining the level of prostate specific antigen (PSA) proteolytic activity in a sample from the subject, the sample being selected from urine, semen, prostatic fluid or post prostatic massage urine and correlating said level of activity to the presence of prostate cancer.

In some embodiments, a method for determining the prognosis of prostate cancer in a subject is provided, the disclosed method encompassing determining the level of enzymatic activity, for example, proteolytic activity, in a sample from said subject wherein the sample is, for example, urine, semen, prostatic fluid, or post prostatic massage urine; normalizing the level of enzymatic activity; and using the normalized enzymatic activity level to prognose prostate cancer.

Thus, in some embodiments, the disclosure provides methods for determining the prognosis of prostate cancer in a subject comprising:

a) contacting a sample taken from a subject with a labeled prostate cancer specific peptide that is a substrate of prostate specific antigen (PSA), said sample selected from the group consisting of urine, semen, prostatic fluid and post prostatic massage urine;

b) determining the level of PSA proteolytic activity in said sample;

c) normalizing said level of proteolytic activity to (i) total PSA in said sample; (ii) total PSA in the serum of said subject; or (iii) prostate volume; and

d) utilizing said normalized proteolytic activity level to prognose prostate cancer in said subject.

In one embodiment, this method further comprises a step of providing said sample prior to determining the level of proteolytic activity in said sample.

In some embodiments, the method for determining the prognosis of prostate cancer in a subject utilizes the PSA proteolytic activity in a sample, said sample being selected from urine, semen, prostatic fluid, or post prostatic massage urine; normalizing PSA enzymatic activity; and using said normalized PSA enzymatic activity to prognose prostate cancer.

In some embodiments, the method for determining the aggressiveness of prostate cancer in a subject utilizes the PSA proteolytic activity in a sample, said sample being selected from urine, semen, prostatic fluid, or post prostatic massage urine; normalizing PSA enzymatic activity; and using said normalized PSA enzymatic activity to prognose prostate cancer.

Thus, in some embodiments, the disclosure provides methods for determining the aggressiveness of prostate cancer in a subject diagnosed with prostate cancer, said method comprising:

a) contacting a sample taken from a subject with a labeled prostate cancer specific peptide that is a substrate of prostate specific antigen (PSA), said sample selected from the group consisting of urine, semen, prostatic fluid and post prostatic massage urine;

b) determining the level of PSA proteolytic activity in said sample;

c) normalizing said level of proteolytic activity to (i) total PSA in said sample; (ii) total PSA in the serum of said subject; or (iii) prostate volume; and

d) utilizing said normalized proteolytic activity level to determine the aggressiveness of said prostate cancer in said subject.

In some embodiments, the method for monitoring the progression of prostate cancer therapy in a subject utilizes the level of PSA proteolytic activity in a sample, said sample being selected from urine, semen, prostatic fluid, or post prostatic massage urine; normalizing PSA enzymatic activity; and using said normalized PSA enzymatic activity to prognose prostate cancer.

Thus, in some embodiments, the disclosure provides methods for monitoring the progression of prostate cancer therapy in a subject comprising:

a) contacting a sample taken from a subject with a labeled prostate cancer specific peptide that is a substrate of prostate specific antigen (PSA), said sample selected from the group consisting of urine, semen, prostatic fluid and post prostatic massage urine;

b) determining the level of PSA proteolytic activity in said sample;

c) normalizing said level of proteolytic activity to (i) total PSA in said sample; (ii) total PSA in the serum of said subject; or (iii) prostate volume; and

d) utilizing said normalized proteolytic activity level to monitor prostate cancer therapy in said subject.

In one embodiment, this method further comprises a step of providing said sample prior to determining the level of proteolytic activity in said sample.

In other embodiments, the methods of disclosure are wherein said labeled prostate cancer specific peptide is labeled HSSKLQ (SEQ ID NO: 1). In some other embodiments, the methods of disclosure are wherein said labeled prostate cancer specific peptide is labeled HSSK-Hiv-Q (SEQ ID NO: 21). In some other embodiments, the methods of disclosure are said labeled prostate cancer specific peptide is labeled HSSK-Hic-Q (SEQ ID NO: 22).

The methods of the disclosure are wherein said label is chromogenic, fluorogenic, or electrochemical.

In particular embodiments, the methods of the disclosure are wherein said labeled prostate cancer specific peptide is fibronectin. In some other embodiments, the methods of the disclosure are wherein said prostate cancer specific peptide ranges from 0.2 mM to 0.4 mM.

### DETAILED DESCRIPTION OF THE INVENTION

The present invention provides a methodology for detecting the presence or absence of cancer with the ability to differentiate between aggressive and non-aggressive prostate cancer. This methodology utilizes the detection of differential enzymatic activity, for example the proteolytic activity of PSA or cleavage of a prostate cancer specific peptide (PCSP), in bodily fluids to in order to classify patients as having aggressive or non-aggressive cancer.

Accordingly, the present invention provides methods for prognosis of cancer, particularly prostate cancer, in a subject. In some cases, distinctions can be drawn between “normal” patients, those significantly free of prostatic disease, cancer patients, and other patients with prostatic conditions such as BPH, as discussed below. In some cases, prognosis may also be done using the methods of the invention.

Typically, prognosis is defined as a prediction of the chance of recovery or survival from a disease, based on statistics of how a disease acts in studies on the general population. However, in this context prognosis is defined as it is related to the disease aggressiveness. As outlined below, aggressiveness and therefore the prognosis of cancer can be determined through enzymatic assay.

One system of grading prostate cancer is the “Gleason Grading System.” The Gleason grading system assigns a grade to each of the two largest areas of cancer in the tissue samples. Grades range from 1 to 5, with 1 being the least aggressive and 5 the most aggressive. Grade 3 tumors, for example, seldom have metastases, but metastases are common with grade 4 or grade 5. The two grades are then added together to produce a Gleason score. A Gleason score of 2 to 4 is considered low grade; 5 through 7, intermediate grade; and 8 through 10, high grade. A tumor with a low Gleason score typically grows slowly enough that it may not pose a significant threat to the patient in his lifetime.

In general, diagnosis in this context is the process of identifying the presence or absence of prostate related disease, particularly prostate cancer. As outlined below, this is done using an enzymatic assay. In some cases, as is more generally outlined below, the results of the protease assay(s) outlined herein can be combined with other factors, including, but not limited to, generally accepted risk factors in prostate cancer nomograms such as prostate size or volume, Gleason scores, serum PSA levels (including various PSA isoforms as well as free PSA), age, lifestyle, etc.

Prostate cancer is a malignant disease of the prostate including, but not limited to, adenocarcinoma, small cell undifferentiated carcinoma and mucinous (colloid) cancer. Prostate cancer can remain localized to the prostate, that is, organ confined, or can spread outside of the prostate. As outlined below, prognosis can be determined through an enzymatic assay for the PSA proteolytic activity.

The disclosure provides methods to quantify PSA proteolytic activity in a sample and associate said PSA proteolytic activity with prostate cancer aggressiveness. This method also assesses the association of prostate cancer aggressiveness with enzymatic activity of PSA in ex vivo prostatic fluid or any other sample matrix.

The examples presented below demonstrate a significant correlation between prostate cancer progression and the enzymatic activity level of PSA in prostatic fluid. The methods provide for the enzymatic activity of uncomplexed PSA in clinically derived samples from prostate cancer patients to establish a diagnostic marker for aggressiveness. The differential in enzymatic activity of PSA between the two populations is a “matrix resultant signal” reported by the PSA-specific probe. Several factors, in concert, affect the enzymatic activity of PSA and result in a particular PSA enzymatic activity level value. Examples of such factors that modulate the enzymatic activity of PSA include zinc concentration, salt concentration, pH, and protease inhibitor concentration. Additionally, PSA levels in prostatic fluid samples are known to be affected by both BPH and carcinoma. Kim, E. D. et al. (1995) Journal of Urology 154, 1802-1805.

Within the non-aggressive group in Example 1, there were 11 samples whose PSA enzymatic activity level values (1238-2626 μg/mL) were greater than the highest PSA enzymatic activity level value measured within the aggressive cohort (1220 μg/mL). This biomarker is distinguished from all others that are positively associated with disease aggressiveness. It suggests that a cutoff value for enzymatic activity of PSA may be established that potentially could eliminate as many as 22% of unnecessary radical prostatectomies within that cohort, with the implication being that the excluded 22% could be managed with active surveillance.

The level of enzymatic activity of PSA in prostatic fluid provides discrimination between clinically aggressive and non-aggressive prostate cancer with an inverse relationship to aggressiveness. This relationship provides a threshold for this biomarker to be tested and clinically validated for establishing cut-offs for further clinical intervention. In one embodiment, monitoring this aggressiveness biomarker is applied for patients currently under active surveillance, eliminating negative outcomes that have been observed with delayed treatment in this population. In one embodiment, assaying expressed prostatic secretions (EPS) (Clark, J. P. et al. (2008) Clinical Chemistry 54, 2007-2017) or post-digital rectal examination (DRE) early catch urine (Hessels, D. et al. (2010) Prostate 70, 10-16) may be used to obtain prostatic fluid-containing samples. In another embodiment, radical prostatectomy samples derived from prostate cancer patients with intermediate aggressive cancer may be used. In yet another embodiment, clinically derived EPS and/or post-DRE early catch urine may be used. The methods of the disclosure may be used alone or with existing diagnostic biomarkers to significantly reduce over-diagnosis and overtreatment of prostate cancer, thus reducing the level of controversy and dissatisfaction of prostate cancer patients world-wide.

In addition to cancer, other diseases of the prostate include, without limitation, benign prostatic hyperplasia (BPH), prostatitis, and prostatic intraepithelial neoplasia (PIN), any or all of which are generally referred to herein as “prostatic disease”.

“Benign prostatic hyperplasia” (“BPH”) is generally used to represent clinical enlargement of the prostate or lower urinary tract symptoms including irritative or obstructed voiding pattern, urinary retention, and frequent urination with an increased residual urine volume. Benign prostatic hypertrophy is reported to occur in over 80% of the male population before the age of 80 years, and that as many as 25% of men reaching age 80 years will require some form of treatment, usually in the form of a surgical procedure (Partin (2000) Benign Prostatic Hyperplasia, in Prostatic Diseases (Lepor H. ed.), W. B. Saunders, Philadelphia, pp 95-105). The cause of BPH remains obscure.

Prostatitis refers to any type of disorder associated with inflammation of the prostate, including chronic and acute bacterial prostatitis and chronic non-bacterial prostatitis, and which is usually associated with symptoms of urinary frequency and/or urinary urgency. A disorder which can mimic the symptoms of prostatitis is prostadynia.

Prostatic intraepithelial neoplasia (PIN) encompasses the various forms and/or degrees of PIN including, but not limited to, high grade prostatic intraepithelial neoplasia and low grade prostatic intraepithelial neoplasia. “HGPIN” refers to high-grade PIN, or “high grade prostatic intraepithelial neoplasia”, while the term “LGPIN” refers to low-grade PIN, or “low grade prostatic intraepithelial neoplasia.”

The present invention provides methods of diagnosing prostatic disease, including cancer and BPH in a male subject, particularly humans, as well as determining the level of aggressiveness and prognosis of prostate cancer if present.

The present methods involve testing samples for proteolytic activity. By “sample” herein is meant a sample containing isoforms of PSA of various enzymatic activity that is correlated to prostatic disease, including, but not limited to, urine, semen, prostatic fluid, seminal vesicle fluid, prostate tissue samples (for example biopsy sample(s) (e.g., homogenized tissue samples) and post prostatic massage urine.

PSA reaches the serum after diffusion from luminal cells through the epithelial basement membrane and prostatic stroma, where it can pass through the capillary basement membrane and epithelial cells or into the lymphatics. (Sokoll et al. 1997). PSA can also be isolated from body fluids including, but not limited to, semen, seminal plasma, prostatatic fluid, serum, urine, urine after prostate massage, and ascites.

Thus, in some embodiments, the sample is urine. In some cases, standard urine is collected, either “first catch” urine or total samples, In some embodiments, urine samples are collected after standard DRE prostatic massage, which are referred to herein as “post prostatic massage urine”.

In other embodiments, the test sample is semen, seminal fluid or seminal plasma. Seminal plasma can be obtained by allowing semen to liquefy for one hour at room temperature followed by centrifugation 1000 g at 4° C. for ten minutes. See e.g., Edstrom A. et al. J. Immunol. 181, 3413-3421 (2008).

In other embodiments, the test sample is prostatic fluid. Prostatic fluid can be obtained following a prostatic massage, i.e. post Digital Rectum Exam (DRE) and either milking the urethra directly (called Expressed Prostatic Secretions, EPS), or in post-DRE urine, or following radical prostatectomy by squeezing the excised prostate gland. In some embodiments, using prostatic fluid collected clinically as EPS is preferred, as post-DRE urine introduces complications to the fluorescent measurement, as it autofluoresces. These complications can be avoided if prostate fluid is used as the test sample since (our studies show) it does not autofluoresce.

In serum, total PSA (tPSA) levels represent the combined concentrations of several free isoforms (fPSA) and protease-inhibitor complexes (cPSA) that can be recognized by immunoassay.

In some embodiments, blood, serum and/or plasma may be used, and in some embodiments, these samples are not preferred.

The samples can be tested either “straight” or directly, with no sample preparation, or with some sample preparation. As will be appreciated by those in the art, a number of sample preparation methods may be utilized, including the removal of cells or non-protease proteins, and buffers (e.g., the addition of high salts, etc.), reagents, or assay components, etc., added.

The present invention provides methods of diagnosing and prognosing subjects using assays for proteolytic activity against a prostate cancer specific peptide (“PCSP”) that correlates with prostatic disease. This assessment of the enzymatic function of PSA in extracted physiological fluids uses a synthetic substrate in a regulated in vitro environment, by providing high excess of a labeled synthetic peptide substrate in-vitro. This synthetic peptide is a substitute model for physiological substrates, such as naturally occurring proteins including macroglobulins and semenogelin. The peptide substrate provides a means of measuring PSA's efficacy as a peptidase in a given physiological sample under standardized conditions, after controlling the ionic activity, the pH and temperature. Examples of the substrates used for the PSA Peptidase Activity are HSSKLQ-Tag or HSSK-Hic-Q-Tag, where Tag represents a label. In some embodiments this label is the AMC (7-amino methyl coumarin) and the products are HSSKLQ and HSSK-Hic-Q respectively. In this context, HIV stands for hydroxyl-isovaleric and HIC stands for hydroxyl-isocaproic. The measurement of the proteolytic function of the enzyme is assessed by measuring the fluorescent signal generated by the cleaved fluorogenic tag.

As shown herein, the presence of prostate cancer can be determined using assays that cleave a PCSP, with greater activity against the peptide correlating to cancer. By “peptides” or grammatical equivalents herein is meant proteins, polypeptides, oligopeptides and peptides, derivatives and analogs, including proteins containing non-naturally occurring amino acids and amino acid analogs, and peptidomimetic structures. The side chains may be in either the (R) or the (S) configuration. In a preferred embodiment, the amino acids are in the (S) or L configuration.

When the peptide is used as a substrate during the assay, e.g., as a PCSP, the peptide can contain both naturally occurring and peptidomimetic structures, as long as the peptidomimetic residues of the PCSP do not interfere with the cleavage of the peptide and/or the correlation of activity to the diagnosis or prognosis.

As discussed below, when the protein is used as a capture substrate it may be desirable in some embodiments to utilize protein analogs to retard degradation by sample contaminants, although in many embodiments capture peptides utilizing native amino acids are used.

Surprisingly, the present invention shows a correlation between the amount of cleavage of PCSPs in samples such as but not limited to expressed prostatic fluid and post prostatic massage urine between prostate cancer patients and BPH and/or control patients, as well as patients with various degrees of aggressiveness and thus can be used in prostate cancer diagnosis, prognosis and therapy monitoring. Thus the invention provides methods of diagnosis and prognosis that rely on the correlation of cleavage of PCSPs with disease state.

Accordingly, the present invention provides substrate peptides that are PCSPs. By “prostate cancer specific peptide” or “PCSP” or “prostatic disease specific peptide” or grammatical equivalents herein is meant a peptide whose cleavage by one or more proteases in a sample is correlated to prostate cancer and disease. In some embodiments, as is more fully outlined below, the PCSP is specific to PSA in the context of the assay. That is, the specificity of the peptide for the protease may be altered depending on what other proteases are present; for example, in general, semen contains more proteases that urine, and thus the absolute specificity of the peptide may be less for urine.

The substrates being used in the present invention depend on the target enzyme. In some embodiments, the enzyme is PSA, as is more fully described below. In the case of PSA, a peptide that finds particular use in the present invention is the peptide HSSKLQ (SEQ ID NO:1), wherein cleavage occurs after the glutamine (Q); see Denmeade et al., Cancer Research 57:4924 (1997), incorporated by reference in its entirety. As outlined below, the PCSPs can be conjugated to labels, including optical (fluorescent) and electrochemical labels, to allow for detection of cleavage.

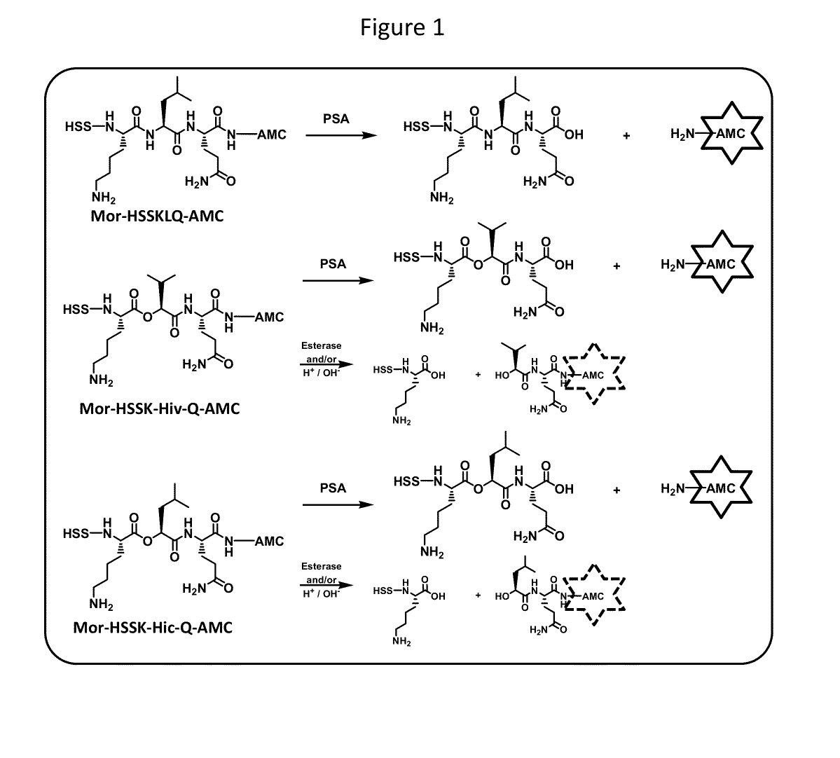

In addition to the HSSKLQ peptide, a number of other peptides are PCSPs, including peptides specific for prostate specific antigen (PSA) serine protease, as further described herein. These peptides include, but are not limited to, for example, some or all of the peptide substrates such as those described in Tables 1, 2, and 3 in Denmeade et al. including, but not limited to, KGISSQY (SEQ ID NO. 2), SRKSQQY (SEQ ID NO. 3), GQKGQHY (SEQ ID NO. 4), EHSSKLQ (SEQ ID NO. 5), QNKISYQ, (SEQ ID NO. 6), ENKISYQ (SEQ ID NO. 7), ATKSKQH (SEQ ID NO. 8), KGLSSQC, (SEQ ID NO. 9), LGGSQQL (SEQ ID NO. 10), QNKGHYQ (SEQ ID NO. 11), TEERQLH (SEQ ID NO. 12), GSFSIQH (SEQ ID NO. 13), SKLQ, as well as analogs. In some embodiments, preferred analogs include, but are not limited to, the substrates shown in FIG. 1, sometimes referred to herein as “AMIDE”, “HIC” and “HIV”. In this context, HIV stands for hydroxyl-isovaleric and HIC stands for hydroxyl-isocaproic. As will be appreciated by those in the art, the peptide sequences listed herein can be modified in a variety of ways, as long as activity is preserved. For example, the peptides shown in FIG. 1 have a morpholino (“mor”) group on the terminal histidine, which is optional. Similarly, the peptides shown in FIG. 1 have 7-Amino-4-methylcoumarin (AMC) as the fluorogenic leaving group, although as outlined herein, a number of other labels can be used. Furthermore, while these peptides are cleaved after the glutamine, Q, depending on the detection system of the assay, it is possible to include additional amino acids at either the N- or C-termini (or both) to this sequence (or the others described herein). That is, as long as there is a measurable change in the signal upon cleavage, e.g. either fluorescence or E⁰, the peptide finds use in the present invention.

Other peptides that find use in the present invention include CHSSLKQK (SEQ ID NO. 14) as described in Zhao et al., Electrochemistry Communications 12:471 (2010); CEEEEHSSLKQKKKK (SEQ ID NO. 15) as described in Roberts et al., JACS 129:11353 (2007); KGISSQY (SEQ ID No. 16) as described in Niemela et al., Clinical Chemistry 48(8):1257 (2002); and a number of peptides described in U.S. Pat. No. 6,265,540 (specifically those in the SEQ ID listings), all of which are hereby incorporated by reference in their entirety.

Such peptides, as well as other enzyme-cleavable peptides, including peptides containing substitute, modified, unnatural or natural amino acids in their sequences, as well as peptides modified at their amino or carboxy terminus, are made from their component amino acids by a variety of methods well known to ordinarily skilled artisans, and practiced thereby using readily available materials and equipment, (see, e.g., The Practice of Peptide Synthesis (2^{nd} Ed.), M. Bodanskzy and A. Bodanskzy, Springer-Verlag, New York, N.Y. (1994), the contents of which are incorporated herein by reference).

These include, for example and without limitation: solid-phase synthesis using the Fmoc protocol (see, e.g., Change and Meieinhofer, Int. J. Pept. Protein Res. 11:246-9 (1978)). Other documents describing peptide synthesis include, for example and without limitation: Miklos Bodansky, Peptide Chemistry, A Practical Textbook 1988, Springer-Verlag, N.Y.; Peptide Synthesis Protocols, Michael W. Pennington and Ben M. Dunn editors, 1994, Humana Press Totowa, N.J.

As described hereinabove, enzyme-cleavable peptides comprise an amino acid sequence which serves as the recognition site for a peptidase capable of cleaving the peptide. The amino acids comprising the enzyme cleavable peptides may include natural, modified, or unnatural amino acids, wherein the natural, modified, or unnatural amino acids may be in either D or L configuration. Natural amino acids include the amino acids alanine, cysteine, aspartic acid, glutamic acid, phenylalanine, glycine, histidine, isoleucine, lysine, leucine, methionine, asparganine, proline, glutamine, arginine, serine, threonine, valine, tryptophan, and tyrosine.

Enzyme-cleavable peptides may also comprise a variety of unnatural or modified amino acids suitable for substitution into the enzyme-cleavable peptide of the invention. A definite list of unnatural amino acids is disclosed in Roberts and Vellaccio, The Peptides, Vol. 5, 341-449 (1983) Academic Press, New York, and is incorporated herein by reference for that purpose. Examples of unnatural or modified amino acids used herein include, without limitation: alpha-amino acid, 2-aminoadipic acid (2-aminohexanedioic acid), alpha-asparagine, 2-aminobutanoic acid or 2-aminobutyric acid .gamma. 4-aminobutyric acid, 2-aminocapric acid (2-aminodecanoic acid), 6-aminocaproic acid, alpha-glutamine, 2-aminoheptanoic acid, 6-aminohexanoic acid, alpha-aminoisobutyric acid (2-aminoalanine), 3-aminoisobutyric acid, beta-alanine, allo-hydroxylysine, allo-isoleucine, 4-amino-7-methylheptanoic acid, 4-amino-5-phenylpentanoic acid, 2-aminopimelic acid (2-aminoheptanedioic acid), gamma-amino-beta-hydroxybenzenepent-anoic acid, 2-aminosuberic acid (2-aminooctanedioic acid), 2-carboxyazetidine, beta-alanine, beta-aspartic acid, Biphenylalanine, 3,6-diaminohexanoic acid (beta-lysine), butanoic acid, 4-amino-3-hydroxybutanoic acid, gamma-amino-beta-hydroxycyclohexanepentanoic acid, cyclobutyl alanine, Cyclohexylalanine, Cyclohexylglycine, N5-aminocarbonylornithine, cyclopentyl alanine, cyclopropyl alanine, 3-sulfoalanine or cysteic acid, 2,4-diaminobutanoic acid, diaminopropionic acid, 2,4-diaminobutyric acid, diphenyl alanine, N,N-dimethylglycine, diaminopimelic acid, 2,3-diaminopropanoic acid or 2,3-diaminopropionic acid, S-ethylthiocysteine, N-ethylasparagine, N-ethylglycine, 4-aza-phenylalanine, 4-fluoro-phenylalanine, gamma-glutamic acid or (γ-E) or (γ-Glu) Gla gamma-carboxyglutamic acid, hydroxyacetic acid (glycolic acid), pyroglutamic acid, homoarginine, homocysteic acid, homocysteine, homohistidine, 2-hydroxyisovaleric acid, homophenylalanine, homoleucine or homo-L homoproline or homo-P homoserine, homoserine, 2-hydroxypentanoic acid, 5-hydroxylysine, 4-hydroxyproline, 2-carboxyoctahydroindole, 3-carboxyisoquinoline, isovaline, 2-hydroxypropanoic acid (lactic acid), mercaptoacetic acid mercaptobutanoic acid, N-methylglycine or sarcosine, 4-methyl-3-hydroxyproline, mercaptopropanoic acid, norleucine, nipecotic acid, nortyrosine, norvaline, omega-amino acid, ornithine, penicillamine (3-mercaptovaline), 2-phenylglycine, 2-carboxypiperidine, sarcosine (N-methylglycine), 2-amino-3-(4-sulfophenyl)propionic acid, 1-amino-1-carboxycyclopentane, statin (4-amino-3-hydroxy-6-methyl heptanoic acid), 3-thienylalanine, epsilon-N-trimethyllysine, 3-thiazolylalanine, thiazolidine 4-carboxylic acid alpha-amino-2,4-dioxopyrimidinepropanoic acid, and 2-naphthylalanine

Enzyme-cleavable peptides may also comprise a variety of modified amino acids wherein an amine or hydroxy function of the amino acid has been chemically modified with an alkyl group, an alkenyl group, a phenyl group, a phenylalkyl group, a heterocyclic group, a heterocyclicalkyl group, a carbocyclic group, or a carbocyclicalkyl group. Examples of chemical modification substituents include, but are not limited to, methyl, ethyl, propyl, butyl, allyl, phenyl, benzyl, pyridyl, pyridylmethyl, and imidazolyl. “The Peptides” Vol 3, 3-88 (1981) discloses numerous suitable sidechain functional groups for modifying amino acids, and is herein incorporated for that purpose.

Examples of modified amino acids include, but are not limited to, N-methylated amino acids, N-methylglycine, N-ethylglycine, N-ethylasparagine, N,N-dimethyllysine, N′-(2-imidazolyl)lysine, O-methyltyrosine, O-benzyltyrosine, O-pyridyltyrosine, O-pyridylmethyltyrosine, O-methylserine, O-t-butylserine, O-allylserine, O-benzylserine, O-methylthreonine, O-t-butylthreonine, O-benzylthreonine, O-methylaspartic acid, O-t-butylaspartic acid, O-benzylaspartic acid, O-methylglutamic acid, O-t-butylglutamic acid, and O-benzylglutamic acid.

Enzyme-cleavable peptides may also comprise a modified amino acid which is 4-azahydroxyphenylalanine (4-azaHof or azaHof), 4-aminomethylalanine, 4-pryidylalanine, 4-azaphenylalanine, morpholinylpropyl glycine, piperazinylpropyl glycine, N-methylpiperazinylpropyl glycine, 4-nitro-hydroxyphenylalanine, 4-hydroxyphenyl glycine, or a 2-(4,6-dimethylpyrimidinyl)lysine.

In some embodiments, fluorogenic PCSPs are utilized. As is known in the art, there are a number of fluorogenic groups that are used in the determination of protease cleavage, including, but not limited to, AMC (7-Amino-4-methylcoumarin); MCA ((7-Methoxycoumarin-4-yl)acetyl), p-nitroanilide (pNA), etc.

In addition to fluorogenic substrates relying on a single fluorophore which is activated by cleavage, fluorescence resonance energy transfer (FRET also known as non-radiative energy transfer or Forster energy transfer) systems can also be used. In these embodiments, a fluorophore reporter and a quencher is used, with the protease cleavage site between the two. As one specific example, the quenching moiety may be a dye molecule capable of quenching the fluorescence of the signal fluorophores via the well-known phenomenon of FRET. In FRET, an excited fluorophore (donor dye; in this instance the signal fluorophore) transfers its excitation energy to another chromophore (acceptor dye; in this instance the quencher). Such a FRET acceptor or quencher may itself be a fluorophore, emitting the transferred energy as fluorescence (fluorogenic FRET quencher or acceptor), or it may be non-fluorescent, emitting the transferred energy by other decay mechanisms (dark FRET quencher or acceptor). Efficient energy transfer depends directly upon the spectral overlap between the emission spectrum of the FRET donor and the absorption spectrum of the FRET quencher or acceptor, as well as the distance between the FRET donor and acceptor). The proximity of the reporter and quencher prior to cleavage results in “quenching”, wherein excitation at the reporter's excitation maxima results in the reporter emitting light at the quencher's excitation wavelength which is absorbed by the quencher molecule, thus resulting in appreciably no detection at the reporter's emission spectra. Upon cleavage, however, the reporter and the quencher are no longer in spatial proximity and thus there is no effective quenching.

Examples of signal and quencher labels that are FRET dye pairs are well known in the art, see for example, Marras et al., 2002, Nucleic Acids Res., 30(21) e122; Wittwer et al., 1997, Biotechniques 22:130-138; Lay and Wittwer, 1997, Clin. Chem. 43:2262-2267; Bernard et al., 1998, Anal. Biochem. 255:101-107; U.S. Pat. Nos. 6,427,156; 6,140,054 and 6,592,847, the disclosures of which are incorporated herein by reference.

In some embodiments, the signal label of the signal probe is a fluorophore and the quencher label of the quencher probe is a moiety capable of quenching the fluorescence signal of the signal fluorophores. Fluorophores are known in the art. Examples of moieties capable of quenching fluorescence signals include Dabcyl, dabsyl BHQ-1, TMR, QSY-7, BHQ-2, black hole Quencher® (Biosearch), and aromatic compounds with nitro or azo groups.

In another specific example, the quenching moiety may be a molecule or chromophore capable of quenching the fluorescence of the signal fluorophore via non-FRET mechanisms. For quenching via collision or direct contact, no spectral overlap between the signal fluorophores and quenching chromophore is required, but the signal fluorophore and quenching chromophore should be in close enough proximity of one another to collide.

In addition, fluorescent based detection systems as discussed above can be done as “solution phase” assays as will be readily appreciated by those in the art. Alternatively, the PSA enzymatic activity tests using fluorescence can be done as “solid support” assays as well. Thus, for example, either a peptide labeled with a single fluorophore as described above or a dual labeled FRET peptide can be attached to a solid support and a test sample can be added and fluorescence monitored.

Similarly, additional amino acids can be incorporated for electrochemical detection as described herein. For example, the electrochemical studies herein, utilize a cysteine after the glutamine for purposes of attaching the peptide to the surface. As will be appreciated in the art, the peptide could be directly attached via a peptide bond to the RAM, or can include additional/different amino acids, including amino acid analogs, as long as the PSA enzyme will still cleave the substrate to produce a signal (e.g., a change in E° or a change in fluorescence).

Thus, other peptides can be used as the capture substrates (e.g., the “PSA peptide”) for use in the assay systems described herein. For example, PSA cleaves with some specificity the peptide HSSKLQ relative, for example, to chymotrypsin. Depending on the test sample, less specific peptides can be used. As will be appreciated those in the art, there are a number of optical (e.g., including fluorescence based) assays that can be run on peptide-based substrates. In general, these rely on optical changes, for example fluorescence, that occur upon cleavage, as generally described above.

Other PSA substrates include naturally occurring substrates such as semenogelin I, semenogelin II, fibronectin, laminin, insulin-like growth factor binding proteins, the single chain form of urokinase-type plasminogen activator and parathyroid hormone related protein.

In general, the cleavage of these PCSPs are correlated to the presence of particular proteases in the samples. Proteases represent a number of families of proteolytic enzymes that catalytically hydrolyze peptide bonds. By “protease” or “proteinase” herein is meant an enzyme that can hydrolyze proteins by hydrolysis of the peptide (amide) bonds that link amino acids. Principal groups of proteases include serine proteases, cysteine proteases, aspartic proteases and metalloproteases.

Serine proteases found in the prostate may be involved in the proteolytic cascade responsible for prostate cancer invasion and metastasis. Two such proteins are urokinase-type plasminogen activator (u-PA) and PSA. Increased synthesis of the protease urokinase has been correlated with an increased ability to metastasize in many cancers. Urokinase activates plasmin from plasminogen which is ubiquitously located in the extracellular space and its activation can cause the degradation of the proteins in the extracellular matrix through which the metastasizing tumor cells invade. Plasmin can also activate the collagenases thus promoting the degradation of the collagen in the basement membrane surrounding the capillaries and lymph system thereby allowing tumor cells to invade into the target tissues Dano et al. (1985) Adv. Cancer. Res., 44: 139.

The present invention provides for the assay of proteases, particularly prostate specific antigen (PSA) serine protease, in the samples. That is, in some embodiments, the activity of PSA in the sample such as post prostatic massage urine or expressed prostatic secretions is assayed using any substrate that is both cleaved by PSA and is not cleaved by other proteases in the particular sample.

Prostate specific antigen (PSA), generally occurring at concentrations of 15-60 μM (that is, 0.5-2 mg/ml), is the most abundant serine protease in prostatic fluid. Prostate specific antigen (PSA) is a ˜33-kDa glycoprotein that shares extensive structural similarity with the glandular kallikrein-like proteinases. Yet, in contrast to the trypsin-like activity common to other kallikreins, PSA appears to manifest chymotrypsin-like activity. The sequence of human PSA is GENBANK: AAD14185; prostate-specific antigen isoform 1 preproprotein (Homo sapiens) is NCBI Reference Sequence: NP_{—}001639 and prostate-specific antigen isoform 3 preproprotein (Homo sapiens) is NCBI Reference Sequence: NP_{—}001025218.

It has been suggested that PSA acts primarily independently as a protease in protein degradation, and not via plasmin, as does u-PA.

PSA is synthesized in the ductal epithelium and prostatic acini and is secreted into the lumina of the prostatic ducts via exocytosis. From the lumen of the prostatic ducts, PSA enters the seminal fluid as it passes through the prostate.

In the seminal fluid are gel-forming proteins, primarily semenogelin I and II and fibronectin, which are produced in the seminal vesicles. These proteins are the major constituents of the seminal coagulum that forms at ejaculation and functions to entrap spermatozoa. PSA functions to liquefy the coagulum and break down the seminal clot through proteolysis of the gel-forming proteins into smaller more soluble fragments, thus releasing the spermatozoa.

Other substrates have been identified and implicate the active PSA isoform in prostate cancer development, including but not limited to, fibronectin, urokinase-type plasminogen activator, insulin-like growth factor binding proteins, latent transforming growth factor-13, and parathyroid hormone-related protein

PSA exists in several free isoforms as well as complexed to protease inhibitors in different biological fluids. Measurement of distinct PSA isoforms has improved the specificity for prostate cancer detection in select populations. Catalona et al. (1998) J. Am. Med. Assoc. 279:1542-1547 and Jansen et al. (2009) Eur. Urol. 55:563-574. Presently, the Hybritech total and free PSA test kits (Beckman Coulter) and the AxSYM® PSA assays (Abbott Laboratories) are among the most widely used for prostate cancer detection in the United States.

The proteolytic action of active PSA though, is not quantified by routine immunoassays. Consequently, assays specific for PSA enzyme activity are desirable as adjuncts for existing tests to ascertain the clinical utility of this important parameter to discriminate benign from malignant disease. Niemela et al. (2002) Clin. Chem. 48:1257-1264, Wu et al. (2004) Clin. Chem. 50:125-129, and Zhu et al. (2006) Biol. Chem. 387:769-772.

Hence, this invention describes the use of diagnostic assays specific for PSA activity to facilitate the identification of potential cancer for eventual inclusion in diagnostic nomograms to inform high-risk patients that biopsy is warranted or to ensure low-risk patients that active surveillance is advisable if not preferable.

This invention also describes the use of enzymatic activity assays specific for PSA to determine the aggressiveness of prostate cancer to assist with monitoring the progress of cancer treatment and provide a prognosis of prostate cancer in a subject, as well as monitor effectiveness of therapy.

The invention encompasses any assay platform (i.e., optical, electrochemical) that specifically detects PSA-triggered peptide cleavage events in samples.

The invention outlined herein show that PSA activity in clinical samples has a significant correlation with cancer-confirmed biopsy results. Therefore, in some embodiments, the invention provides a method of diagnosing, prognosing, or monitoring the progression of prostate cancer therapies (including, but not limited to, chemotherapeutic treatment and radiation treatment, including brachytherapy and external beam radiation, as well as other types of radiation or beam therapies). The method includes measuring the enzymatic activity of PSA in samples from patients; normalizing enzymatic activity; and using that normalized measure to diagnose, prognose, and/or monitor the progression of prostate cancer therapies.

In general, diagnosis and prognosis may be done by comparing the results to PSA activity levels of normal patients, such that increased PSA activity is a marker for the presence of prostate cancer. After diagnosis therapy may be monitored by taking repeated measurements of patients undergoing treatment, over time, to monitor the PSA activity levels to be vigilant for decreasing levels of enzymatic activity in patients, which have been found to be correlated with increase tumor volume, metastasis, or increasing aggressiveness. The lack of change over time may also allow physicians to maintain or augment therapies as indicated.

As will also be appreciated by those in the art, labels in addition to the optical labels described above and the electrochemical labels outlined below can also be used.

As outlined herein, optical (e.g., fluorescent) assays may be done using any number of known formats. Samples can be run independently or in batches, using any number of systems, including robotic systems, etc.

In one aspect, the present invention provides methods for detecting an enzyme such as PSA in a test sample using an electrochemical assay. The general system is described in U.S. Ser. Nos. 60/980,733; 12/253,828; 61/087,094; 12/253,875; and 61/087,102; all of which are expressly incorporated by reference in their entirety, and in particular for the components of the invention.

As will be appreciated by those in the art, the components of the assay systems described herein can be independently included and excluded in the final system, such that different combinations of components of the invention can be used. The electrochemical assay may encompass an electrode which includes, without limitation, a self-assembled monolayer (SAM) and a covalently attached electroactive moiety (EAM, also referred to herein as a “redox active molecule complex” (ReAMC)).

By “electrode” is meant a composition, which, when connected to an electronic device, is able to sense a current or charge and convert it to a signal. Preferred electrodes are known in the art and include, but are not limited to, certain metals and their oxides, including gold; platinum; palladium; silicon; aluminum; metal oxide electrodes including platinum oxide, titanium oxide, tin oxide, indium tin oxide, palladium oxide, silicon oxide, aluminum oxide, molybdenum oxide (Mo₂O₆), tungsten oxide (WO₃) and ruthenium oxides; and carbon (including glassy carbon electrodes, graphite and carbon paste). Preferred electrodes include gold, silicon, carbon and metal oxide electrodes, with gold being particularly preferred.

The EAM comprises a transition metal complex with a first E°. Also attached to the electrode is a plurality of enzyme substrates (“capture substrates”, sometimes also referred to herein as “PSA substrates” or “PSA peptides” when the target enzyme is PSA) of the target enzyme.

Thus, in this method, the test sample is added to the electrode, the target enzyme and the substrates of the target enzymes form a plurality of reactants. The presence of the enzyme is determined by measuring a change of the E°, resulting from a change in the environment of the EAM.

In one aspect, the present invention provides ligand architectures attached to an electrode.

In some embodiments, the capture substrate provides a coordination atom; in others, while the ReAMC is a single molecule attached to the electrode, the capture substrate does not provide a coordination atom. In other embodiments, there is no ReAMC; rather the EAM and the capture substrate are attached separately to the electrode.

As is described further below several different geometries can be used in the present invention. In one embodiment, the EAM also includes a capture substrate, forming what is referred to herein as a “redox active moiety complex” or ReAMC.

The electrodes described herein are depicted as a flat surface, which is only one of the possible conformations of the electrode and is for schematic purposes only. The conformation of the electrode will vary with the detection method used.

For example, flat planar electrodes may be preferred for optical detection methods, or when arrays of peptides are made, thus requiring addressable locations for both synthesis and detection. Alternatively, for single probe analysis, the electrode may be in the form of a tube, with the components of the system such as SAMs, EAMs and capture ligands bound to the inner surface. This allows a maximum of surface area containing the nucleic acids to be exposed to a small volume of sample.

The electrodes of the invention are generally incorporated into biochip cartridges and can take a wide variety of configurations, and can include working and reference electrodes, interconnects (including “through board” interconnects), and microfluidic components. See, for example U.S. Pat. No. 7,312,087, incorporated herein by reference in its entirety.

The biochip cartridges include substrates comprising the arrays of biomolecules, and can be configured in a variety of ways. For example, the chips can include reaction chambers with inlet and outlet ports for the introduction and removal of reagents. In addition, the cartridges can include caps or lids that have microfluidic components, such that the sample can be introduced, reagents added, reactions done, and then the sample is added to the reaction chamber comprising the array for detection.

In a preferred embodiment, the biochips comprise substrates with a plurality of array locations. By “substrate” or “solid support” or other grammatical equivalents herein is meant any material that can be modified to contain discrete individual sites appropriate of the attachment or association of capture ligands.

Suitable substrates include metal surfaces such as gold, electrodes as defined below, glass and modified or functionalized glass, fiberglass, teflon, ceramics, mica, plastic (including acrylics, polystyrene and copolymers of styrene and other materials, polypropylene, polyethylene, polybutylene, polyimide, polycarbonate, polyurethanes, Teflon™, and derivatives thereof, etc.), GETEK (a blend of polypropylene oxide and fiberglass), etc, polysaccharides, nylon or nitrocellulose, resins, silica or silica-based materials including silicon and modified silicon, carbon, metals, inorganic glasses and a variety of other polymers, with printed circuit board (PCB) and polyethylene terphtalate (PET) materials being particularly preferred.

The present system finds particular utility in array formats, i.e., wherein there is a matrix of addressable detection electrodes (herein generally referred to “pads”, “addresses” or “micro-locations”). By “array” herein is meant a plurality of capture ligands in an array format; the size of the array will depend on the composition and end use of the array. Arrays containing from about 2 different capture substrates to many thousands can be made.

In a preferred embodiment, the detection electrodes are formed on a substrate. In addition, the discussion herein is generally directed to the use of gold electrodes, but as will be appreciated by those in the art, other electrodes can be used as well. The substrate can comprise a wide variety of materials, as outlined herein and in the cited references.

In general, preferred materials include printed circuit board materials. Circuit board materials are those that comprise an insulating substrate that is coated with a conducting layer and processed using lithography techniques, particularly photolithography techniques, to form the patterns of electrodes and interconnects (sometimes referred to in the art as interconnections or leads). The insulating substrate is generally, but not always, a polymer.

As is known in the art, one or a plurality of layers may be used, to make either “two dimensional” (e.g., all electrodes and interconnections in a plane) or “three dimensional” (wherein the electrodes are on one surface and the interconnects may go through the board to the other side or wherein electrodes are on a plurality of surfaces) boards. Three dimensional systems frequently rely on the use of drilling or etching, followed by electroplating with a metal such as copper, such that the “through board” interconnections are made. Circuit board materials are often provided with a foil already attached to the substrate, such as a copper foil, with additional copper added as needed (for example for interconnections), for example by electroplating. The copper surface may then need to be roughened, for example through etching, to allow attachment of the adhesion layer.

Accordingly, in a preferred embodiment, the present invention provides biochips (sometimes referred to herein “chips”) that comprise substrates comprising a plurality of electrodes, preferably gold electrodes. The number of electrodes is as outlined for arrays. Each electrode preferably comprises a self-assembled monolayer as outlined herein. In a preferred embodiment, one of the monolayer-forming species comprises a capture ligand as outlined herein. In addition, each electrode has an interconnection that is attached to the electrode at one end and is ultimately attached to a device that can control the electrode. That is, each electrode is independently addressable.

Finally, the compositions of the invention can include a wide variety of additional components, including microfluidic components and robotic components (see for example U.S. Pat. Nos. 6,942,771 and 7,312,087 and related cases, both of which are hereby incorporated by reference in its entirety), and detection systems including computers utilizing signal processing techniques (see for example U.S. Pat. No. 6,740,518, hereby incorporated by reference in its entirety.

Self Assembled Monolayer Spacers

In some embodiments, the electrodes optionally further comprise a SAM. By “monolayer” or “self-assembled monolayer” or “SAM” herein is meant a relatively ordered assembly of molecules spontaneously chemisorbed on a surface, in which the molecules are oriented approximately parallel to each other and roughly perpendicular to the surface. Each of the molecules includes a functional group that adheres to the surface, and a portion that interacts with neighboring molecules in the monolayer to form the relatively ordered array.

A “mixed” monolayer comprises a heterogeneous monolayer, that is, where at least two different molecules make up the monolayer. As outlined herein, the use of a monolayer reduces the amount of non-specific binding of biomolecules to the surface, and, in the case of nucleic acids, increases the efficiency of oligonucleotide hybridization as a result of the distance of the oligonucleotide from the electrode. Thus, a monolayer facilitates the maintenance of the target enzyme away from the electrode surface.

In addition, a monolayer serves to keep charge carriers away from the surface of the electrode. Thus, this layer helps to prevent electrical contact between the electrodes and the ReAMCs, or between the electrode and charged species within the solvent. Such contact can result in a direct “short circuit” or an indirect short circuit via charged species which may be present in the sample. Accordingly, the monolayer is preferably tightly packed in a uniform layer on the electrode surface, such that a minimum of “holes” exist. The monolayer thus serves as a physical barrier to block solvent accessibility to the electrode.

In some embodiments, the monolayer comprises conductive oligomers. By “conductive oligomer” herein is meant a substantially conducting oligomer, preferably linear, some embodiments of which are referred to in the literature as “molecular wires”. By “substantially conducting” herein is meant that the oligomer is capable of transferring electrons at 100 Hz.

Generally, the conductive oligomer has substantially overlapping π-orbitals, i.e., conjugated i-orbitals, as between the monomeric units of the conductive oligomer, although the conductive oligomer may also contain one or more sigma (σ) bonds. Additionally, a conductive oligomer may be defined functionally by its ability to inject or receive electrons into or from an associated EAM. Furthermore, the conductive oligomer is more conductive than the insulators as defined herein. Additionally, the conductive oligomers of the invention are to be distinguished from electroactive polymers, that themselves may donate or accept electrons.

A more detailed description of conductive oligomers is found in WO/1999/57317, herein incorporated by reference in its entirety. In particular, the conductive oligomers as shown in Structures 1 to 9 on page 14 to 21 of WO/1999/57317 find use in the present invention. In some embodiments, the conductive oligomer has the following structure:

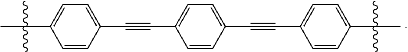 [SMILES: *c1ccc(C#Cc2ccc(C#Cc3ccc(*)cc3)cc2)cc1]

In addition, the terminus of at least some of the conductive oligomers in the monolayer is electronically exposed. By “electronically exposed” herein is meant that upon the placement of an EAM in close proximity to the terminus, and after initiation with the appropriate signal, a signal dependent on the presence of the EAM may be detected. The conductive oligomers may or may not have terminal groups. Thus, in a preferred embodiment, there is no additional terminal group, and the conductive oligomer terminates with a terminal group; for example, such as an acetylene bond.

Alternatively, in some embodiments, a terminal group is added, sometimes depicted herein as “Q”. A terminal group may be used for several reasons; for example, to contribute to the electronic availability of the conductive oligomer for detection of EAMs, or to alter the surface of the SAM for other reasons; for example, to prevent non-specific binding. For example, there may be negatively charged groups on the terminus to form a negatively charged surface such that when the target analyte is a peptide as defined herein that will allow for binding of the protease PSA, followed by specific cleavage of the peptide. Preferred terminal groups include —NH₂, —OH, —COOH, and alkyl groups such as —CH₃, and (poly)alkyloxides such as (poly)ethylene glycol, with —OCH₂CH₂OH, —(OCH₂CH₂O)₂H, —(OCH₂CH₂O)₃H, and —(OCH₂CH₂O)₄H being preferred.

In one embodiment, it is possible to use mixtures of conductive oligomers with different types of terminal groups. Thus, for example, some of the terminal groups may facilitate detection, and some may prevent non-specific binding.

In some embodiments, the electrode further comprises a passivation agent, preferably in the form of a monolayer on the electrode surface. For some analytes the efficiency of analyte binding (i.e., transitory binding of the protease and subsequent cleavage) may increase when the binding ligand is at a distance from the electrode. In addition, the presence of a monolayer can decrease non-specific binding to the surface (which can be further facilitated by the use of a terminal group). A passivation agent layer facilitates the maintenance of the binding ligand and/or analyte away from the electrode surface. In addition, a passivation agent serves to keep charge carriers away from the surface of the electrode. Thus, this layer helps to prevent electrical contact between the electrodes and the electron transfer moieties, or between the electrode and charged species within the solvent. Such contact can result in a direct “short circuit” or an indirect short circuit via charged species which may be present in the sample.

Accordingly, the monolayer of passivation agents is preferably tightly packed in a uniform layer on the electrode surface, such that a minimum of “holes” exist. Alternatively, the passivation agent may not be in the form of a monolayer, but may be present to help the packing of the conductive oligomers or other characteristics.

The passivation agents thus serve as a physical barrier to block solvent accessibility to the electrode. As such, the passivation agents themselves may in fact be either (1) conducting or (2) nonconducting, i.e. insulating, molecules. Thus, in one embodiment, the passivation agents are conductive oligomers, as described herein, with or without a terminal group to block or decrease the transfer of charge to the electrode. Other passivation agents which may be conductive include oligomers of —CF₂)ₙ—, —CHF)ₙ— and —(CFR)ₙ—. In a preferred embodiment, the passivation agents are insulator moieties.

In some embodiments, the monolayers comprise insulators. An “insulator” is a substantially nonconducting oligomer, preferably linear. By “substantially nonconducting” herein is meant that the rate of electron transfer through the insulator is slower than the rate of electron transfer through the conductive oligomer. Stated differently, the electrical resistance of the insulator is higher than the electrical resistance of the conductive oligomer. It should be noted however that even oligomers generally considered to be insulators, such as —(CH2)16 molecules, still may transfer electrons, albeit at a slow rate.

In some embodiments, the insulators have a conductivity, S, of about 10-7 Ω⁻¹ cm⁻¹ or lower, with less than about 10⁻⁸ Ω⁻¹ cm⁻¹ being preferred. Gardner et al., Sensors and Actuators A 51 (1995) 57-66, incorporated herein by reference.

Generally, insulators are alkyl or heteroalkyl oligomers or moieties with sigma bonds, although any particular insulator molecule may contain aromatic groups or one or more conjugated bonds. By “heteroalkyl” herein is meant an alkyl group that has at least one heteroatom, i.e. nitrogen, oxygen, sulfur, phosphorus, silicon or boron included in the chain. Alternatively, the insulator may be quite similar to a conductive oligomer with the addition of one or more heteroatoms or bonds that serve to inhibit or slow, preferably substantially, electron transfer. In some embodiments the insulator comprises C₆-C₁₆ alkyl.

The passivation agents, including insulators, may be substituted with R groups as defined herein to alter the packing of the moieties or conductive oligomers on an electrode, the hydrophilicity or hydrophobicity of the insulator, and the flexibility, i.e., the rotational, torsional or longitudinal flexibility of the insulator. For example, branched alkyl groups may be used. In addition, the terminus of the passivation agent, including insulators, may contain an additional group to influence the exposed surface of the monolayer, sometimes referred to herein as a terminal group (“TG”). For example, the addition of charged, neutral or hydrophobic groups may be done to inhibit non-specific binding from the sample, or to influence the kinetics of binding of the analyte, etc. For example, there may be charged groups on the terminus to form a charged surface to encourage or discourage binding of certain target analytes or to repel or prevent from lying down on the surface.

The length of the passivation agent will vary as needed. Generally, the length of the passivation agents is similar to the length of the conductive oligomers, as outlined above. In addition, the conductive oligomers may be basically the same length as the passivation agents or longer than them, resulting in the binding ligands being more accessible to the solvent.

The monolayer may comprise a single type of passivation agent, including insulators, or different types.

Suitable insulators are known in the art, and include, but are not limited to, —(CH₂)ₙ—, —(CRH)ₙ—, and —(CR₂)ₙ—, ethylene glycol or derivatives using other heteroatoms in place of oxygen, i.e. nitrogen or sulfur (sulfur derivatives are not preferred when the electrode is gold). In some embodiments, the insulator comprises C₆ to C₁₆ alkyl.

In some embodiments, the electrode is a metal surface and need not necessarily have interconnects or the ability to do electrochemistry.

Anchor Groups

The present invention provides compounds comprising an anchor group. By “anchor” or “anchor group” herein is meant a chemical group that attaches the compounds of the invention to an electrode.

As will be appreciated by those in the art, the composition of the anchor group will vary depending on the composition of the surface to which it is attached. In the case of gold electrodes, both pyridinyl anchor groups and thiol based anchor groups find particular use.

The covalent attachment of the conductive oligomer may be accomplished in a variety of ways, depending on the electrode and the conductive oligomer used. Generally, some type of linker is used, as depicted below as “A” in Structure 1, where X is the conductive oligomer, and the hatched surface is the electrode:

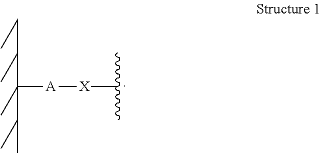

In this embodiment, “A” is a linker or atom. The choice of “A” will depend in part on the characteristics of the electrode. Thus, for example, “A” may be a sulfur moiety when a gold electrode is used. Alternatively, when metal oxide electrodes are used, “A” may be a silicon (silane) moiety attached to the oxygen of the oxide (see, for example, Chen et al., Langmuir 10:3332-3337 (1994); Lenhard et al., J. Electroanal. Chem. 78:195-201 (1977), both of which are expressly incorporated by reference). When carbon based electrodes are used, A may be an amino moiety (preferably a primary amine; see for example Deinhammer et al., Langmuir 10:1306-1313 (1994)). Thus, preferred “A” moieties include, but are not limited to, silane moieties, sulfur moieties (including alkyl sulfur moieties), and amino moieties.

In some embodiments, the electrode is a carbon electrode, i.e. a glassy carbon electrode, and attachment is via a nitrogen of an amine group. A representative structure is depicted in Structure 15 of US Patent Application Publication No. 20080248592, hereby incorporated by reference in its entirety but particularly for Structures as described therein and the description of different anchor groups and the accompanying text. Again, additional atoms may be present, i.e., linkers and/or terminal groups.

In Structure 16 of US Patent Application Publication No. 20080248592, hereby incorporated by reference as above, the oxygen atom is from the oxide of the metal oxide electrode. The Si atom may also contain other atoms, i.e., be a silicon moiety containing substitution groups. Other attachments for SAMs to other electrodes are known in the art; see for example Napier et al., Langmuir, 1997, for attachment to indium tin oxide electrodes, and also the chemisorption of phosphates to an indium tin oxide electrode (talk by H. Holden Thorpe, CHI conference, May 4-5, 1998).

In one preferred embodiment, indium-tin-oxide (ITO) is used as the electrode, and the anchor groups are phosphonate-containing species.

Sulfur Anchor Groups

Although depicted in Structure 1 as a single moiety, the conductive oligomer may be attached to the electrode with more than one “A” moiety; the “A” moieties may be the same or different. Thus, for example, when the electrode is a gold electrode, and “A” is a sulfur atom or moiety, multiple sulfur atoms may be used to attach the conductive oligomer to the electrode, such as is generally depicted below in Structures 2, 3 and 4. As will be appreciated by those in the art, other such structures can be made. In Structures 2, 3 and 4 the A moiety is just a sulfur atom, but substituted sulfur moieties may also be used.

Thus, for example, when the electrode is a gold electrode, and “A” is a sulfur atom or moiety, such as generally depicted below in Structure 6, multiple sulfur atoms may be used to attach the conductive oligomer to the electrode, such as is generally depicted below in Structures 2, 3 and 4. As will be appreciated by those in the art, other such structures can be made. In Structures 2, 3 and 4, the “A” moiety is just a sulfur atom, but substituted sulfur moieties may also be used.

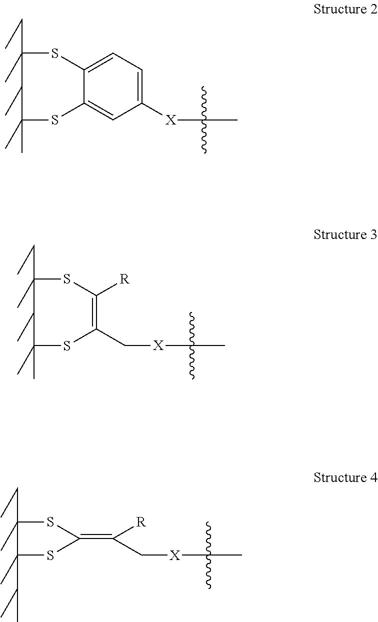

It should also be noted that similar to Structure 4, it may be possible to have a conductive oligomer terminating in a single carbon atom with three sulfur moieties attached to the electrode.

In another aspect, the present invention provide anchor comprise conjugated thiols. Some exemplary complexes are with conjugated thiol anchors. In some embodiments, the anchor comprises an alkylthiol group. The two compounds are based on carbene and 4-pyridylalanine, respectively.

In another aspect, the present invention provides conjugated multipodal thio-containing compounds that serve as anchoring groups in the construction of electroactive moieties for analyte detection on electrodes, such as gold electrodes. That is, spacer groups (which can be attached to EAMs, ReAMCs, or an “empty” monolayer forming species) are attached using two or more sulfur atoms. These mulitpodal anchor groups can be linear or cyclic, as described herein.

In some embodiments, the anchor groups are “bipodal”, containing two sulfur atoms that will attach to the gold surface, and linear, although in some cases it can be possible to include systems with other multipodalities (e.g., “tripodal”). Such a multipodal anchoring group display increased stability and/or allow a greater footprint for preparing SAMs from thiol-containing anchors with sterically demanding headgroups.

In some embodiments, the anchor comprises cyclic disulfides (“bipod”). Although in some cases it can be possible to include ring system anchor groups with other multipodalities (e.g., “tripodal”). The number of the atoms of the ring can vary, for example from 5 to 10, and also includes multicyclic anchor groups, as discussed below

In some embodiments, the anchor groups comprise a [1,2,5]-dithiazepane unit which is seven-membered ring with an apex nitrogen atom and a intramolecular disulfide bond as shown below:

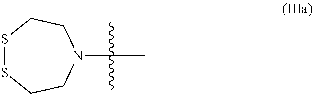 [SMILES: *N1CCSSCC1]

In Structure (IIIa), it should also be noted that the carbon atoms of the ring can additionally be substituted. As will be appreciated by those in the art, other membered rings are also included. In addition, multicyclic ring structures can be used, which can include cyclic heteroalkanes such as the [1,2,5]-dithiazepane shown above substituted with other cyclic alkanes (including cyclic heteroalkanes) or aromatic ring structures.

In some embodiments, the anchor group and part of the spacer has the structure shown below

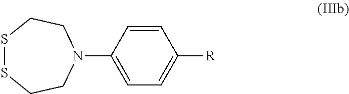

The “R” group herein can be any substitution group, including a conjugated oligophenylethynylene unit with terminal coordinating ligand for the transition metal component of the EAM.

The anchors are synthesized from a bipodal intermediate (I) (the compound as formula III where R═I), which is described in Li et al., Org. Lett. 4:3631-3634 (2002), herein incorporated by reference. See also Wei et al., J. Org, Chem. 69:1461-1469 (2004), herein incorporated by reference.

The number of sulfur atoms can vary as outlined herein, with particular embodiments utilizing one, two, and three per spacer.

Electroactive Moieties

In addition to anchor groups, the present invention provides compound comprising electroactive moieties. By “electroactive moiety (EAM)” or “transition metal complex” or “redox active molecule” or “electron transfer moiety (ETM)” herein is meant a metal-containing compound which is capable of reversibly or semi-reversibly transferring one or more electrons. It is to be understood that electron donor and acceptor capabilities are relative; that is, a molecule which can lose an electron under certain experimental conditions will be able to accept an electron under different experimental conditions.

It is to be understood that the number of possible transition metal complexes is very large, and that one skilled in the art of electron transfer compounds will be able to utilize a number of compounds in the present invention. By “transitional metal” herein is meant metals whose atoms have a partial or completed shell of electrons. Suitable transition metals for use in the invention include, but are not limited to, cadmium (Cd), copper (Cu), cobalt (Co), palladium (Pd), zinc (Zn), iron (Fe), ruthenium (Ru), rhodium (Rh), osmium (Os), rhenium (Re), platinum (Pt), scandium (Sc), titanium (Ti), vanadium (V), chromium (Cr), manganese (Mn), nickel (Ni), molybdenum (Mo), technetium (Tc), tungsten (W), and iridium (Ir). That is, the first series of transition metals, the platinum metals (Ru, Rh, Pd, Os, Ir and Pt), along with Fe, Re, W, Mo and Tc, find particular use in the present invention. Particularly preferred are metals that do not change the number of coordination sites upon a change in oxidation state, including ruthenium, osmium, iron, platinum and palladium, with osmium, ruthenium and iron being especially preferred, and osmium finding particular use in many embodiments. In some embodiments, iron is not preferred. Generally, transition metals are depicted herein as TM or M.

The transition metal and the coordinating ligands form a metal complex. By “ligand” or “coordinating ligand” (depicted herein in the figures as “L”) herein is meant an atom, ion, molecule, or functional group that generally donates one or more of its electrons through a coordinate covalent bond to, or shares its electrons through a covalent bond with, one or more central atoms or ions.

The other coordination sites of the metal are used for attachment of the transition metal complex to either a capture ligand (directly or indirectly using a linker), or to the electrode (frequently using a spacer, as is more fully described below), or both. Thus for example, when the transition metal complex is directly joined to a binding ligand, one, two or more of the coordination sites of the metal ion may be occupied by coordination atoms supplied by the binding ligand (or by the linker, if indirectly joined). In addition, or alternatively, one or more of the coordination sites of the metal ion may be occupied by a spacer used to attach the transition metal complex to the electrode. For example, when the transition metal complex is attached to the electrode separately from the binding ligand as is more fully described below, all of the coordination sites of the metal (n) except 1 (n−1) may contain polar ligands.

Suitable small polar ligands, generally depicted herein as “L”, fall into two general categories, as is more fully described herein. In one embodiment, the small polar ligands will be effectively irreversibly bound to the metal ion, due to their characteristics as generally poor leaving groups or as good sigma donors, and the identity of the metal. These ligands may be referred to as “substitutionally inert”. Alternatively, as is more fully described below, the small polar ligands may be reversibly bound to the metal ion, such that upon binding of a target analyte, the analyte may provide one or more coordination atoms for the metal, effectively replacing the small polar ligands, due to their good leaving group properties or poor sigma donor properties. These ligands may be referred to as “substitutionally labile”. The ligands preferably form dipoles, since this will contribute to a high solvent reorganization energy.

Some of the structures of transitional metal complexes are shown below:

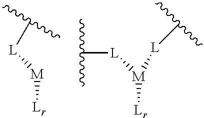

L are the co-ligands, that provide the coordination atoms for the binding of the metal ion. As will be appreciated by those in the art, the number and nature of the co-ligands will depend on the coordination number of the metal ion. Mono-, di- or polydentate co-ligands may be used at any position. Thus, for example, when the metal has a coordination number of six, the L from the terminus of the conductive oligomer, the L contributed from the nucleic acid, and r, add up to six. Thus, when the metal has a coordination number of six, r may range from zero (when all coordination atoms are provided by the other two ligands) to four, when all the co-ligands are monodentate. Thus generally, r will be from 0 to 8, depending on the coordination number of the metal ion and the choice of the other ligands.

In one embodiment, the metal ion has a coordination number of six and both the ligand attached to the conductive oligomer and the ligand attached to the nucleic acid are at least bidentate; that is, r is preferably zero, one (i.e. the remaining co-ligand is bidentate) or two (two monodentate co-ligands are used).

As will be appreciated in the art, the co-ligands can be the same or different. Suitable ligands fall into two categories: ligands which use nitrogen, oxygen, sulfur, carbon or phosphorus atoms (depending on the metal ion) as the coordination atoms (generally referred to in the literature as sigma (σ) donors) and organometallic ligands such as metallocene ligands (generally referred to in the literature as pi (π) donors, and depicted herein as Lm). Suitable nitrogen donating ligands are well known in the art and include, but are not limited to, cyano (C≡N), NH₂; NHR; NRR′; pyridine; pyrazine; isonicotinamide; imidazole; bipyridine and substituted derivatives of bipyridine; terpyridine and substituted derivatives; phenanthrolines, particularly 1,10-phenanthroline (abbreviated phen) and substituted derivatives of phenanthrolines such as 4,7-dimethylphenanthroline and dipyridol[3,2-a:2′,3′-c]phenazine (abbreviated dppz); dipyridophenazine; 1,4,5,8,9,12-hexaazatriphenylene (abbreviated hat); 9,10-phenanthrenequinone diimine (abbreviated phi); 1,4,5,8-tetraazaphenanthrene (abbreviated tap); 1,4,8,11-tetra-azacyclotetradecane (abbreviated cyclam) and isocyanide. Substituted derivatives, including fused derivatives, may also be used. In some embodiments, porphyrins and substituted derivatives of the porphyrin family may be used. See for example, Comprehensive Coordination Chemistry, Ed. Wilkinson et al., Pergammon Press, 1987, Chapters 13.2 (pp 73-98), 21.1 (pp. 813-898) and 21.3 (pp 915-957), all of which are hereby expressly incorporated by reference.

As will be appreciated in the art, any ligand donor(1)-bridge-donor(2) where donor (1) binds to the metal and donor(2) is available for interaction with the surrounding medium (solvent, protein, etc) can be used in the present invention, especially if donor(1) and donor(2) are coupled through a pi system, as in cyanos (C is donor(1), N is donor(2), pi system is the CN triple bond). One example is bipyrimidine, which looks much like bipyridine but has N donors on the “back side” for interactions with the medium. Additional co-ligands include, but are not limited to cyanates, isocyanates (—N═C═O), thiocyanates, isonitrile, N₂, O₂, carbonyl, halides, alkoxyide, thiolates, amides, phosphides, and sulfur containing compound such as sulfino, sulfonyl, sulfoamino, and sulfamoyl.

In some embodiments, multiple cyanos are used as co-ligand to complex with different metals. For example, seven cyanos bind Re(III); eight bind Mo(IV) and W(IV). Thus at Re(III) with 6 or less cyanos and one or more L, or Mo(IV) or W(IV) with 7 or less cyanos and one or more L can be used in the present invention. The EAM with W(IV) system has particular advantages over the others because it is more inert, easier to prepare, more favorable reduction potential. Generally that a larger CN/L ratio will give larger shifts.

Suitable sigma donating ligands using carbon, oxygen, sulfur and phosphorus are known in the art. For example, suitable sigma carbon donors are found in Cotton and Wilkenson, Advanced Organic Chemistry, 5^{th} Edition, John Wiley & Sons, 1988, hereby incorporated by reference; see page 38, for example. Similarly, suitable oxygen ligands include crown ethers, water and others known in the art. Phosphines and substituted phosphines are also suitable; see page 38 of Cotton and Wilkenson.

The oxygen, sulfur, phosphorus and nitrogen-donating ligands are attached in such a manner as to allow the heteroatoms to serve as coordination atoms.

In some embodiments, organometallic ligands are used. In addition to purely organic compounds for use as redox moieties, and various transition metal coordination complexes with δ-bonded organic ligand with donor atoms as heterocyclic or exocyclic substituents, there is available a wide variety of transition metal organometallic compounds with π-bonded organic ligands (see Advanced Inorganic Chemistry, 5^{th} Ed., Cotton & Wilkinson, John Wiley & Sons, 1988, chapter 26; Organometallics, A Concise Introduction, Elschenbroich et al., 2^{nd} Ed., 1992, VCH; and Comprehensive Organometallic Chemistry II, A Review of the Literature 1982-1994, Abel et al. Ed., Vol. 7, chapters 7, 8, 10 & 11, Pergamon Press, hereby expressly incorporated by reference). Such organometallic ligands include cyclic aromatic compounds such as the cyclopentadienide ion [C₅H₅ (−1)] and various ring substituted and ring fused derivatives, such as the indenylide (−1) ion, that yield a class of bis(cyclopentadieyl)metal compounds, (i.e., the metallocenes); see, for example Robins et al., J. Am. Chem. Soc. 104:1882-1893 (1982); and Gassman et al., J. Am. Chem. Soc. 108:4228-4229 (1986), incorporated by reference. Of these, ferrocene [(C₅H₅)₂Fe] and its derivatives are prototypical examples which have been used in a wide variety of chemical (Connelly et al., Chem. Rev. 96:877-910 (1996), incorporated by reference) and electrochemical (Geiger et al., Advances in Organometallic Chemistry 23:1-93; and Geiger et al., Advances in Organometallic Chemistry 24:87, incorporated by reference) electron transfer or “redox” reactions. Metallocene derivatives of a variety of the first, second and third row transition metals are potential candidates as redox moieties that are covalently attached to either the ribose ring or the nucleoside base of nucleic acid.

Other potentially suitable organometallic ligands include cyclic arenes such as benzene, to yield bis(arene)metal compounds and their ring substituted and ring fused derivatives, of which bis(benzene)chromium is a prototypical example. Other acyclic π-bonded ligands such as the allyl(−1) ion, or butadiene yield potentially suitable organometallic compounds, and all such ligands, in conduction with other π-bonded and δ-bonded ligands constitute the general class of organometallic compounds in which there is a metal to carbon bond. Electrochemical studies of various dimers and oligomers of such compounds with bridging organic ligands, and additional non-bridging ligands, as well as with and without metal-metal bonds are potential candidate redox moieties in nucleic acid analysis.

When one or more of the co-ligands is an organometallic ligand, the ligand is generally attached via one of the carbon atoms of the organometallic ligand, although attachment may be via other atoms for heterocyclic ligands. Preferred organometallic ligands include metallocene ligands, including substituted derivatives and the metalloceneophanes (see page 1174 of Cotton and Wilkenson, supra). For example, derivatives of metallocene ligands such as methylcyclopentadienyl, with multiple methyl groups being preferred, such as pentamethylcyclopentadienyl, can be used to increase the stability of the metallocene. In a preferred embodiment, only one of the two metallocene ligands of a metallocene are derivatized.

As described herein, any combination of ligands may be used. Preferred combinations include: a) all ligands are nitrogen donating ligands; b) all ligands are organometallic ligands; and c) the ligand at the terminus of the conductive oligomer is a metallocene ligand and the ligand provided by the nucleic acid is a nitrogen donating ligand, with the other ligands, if needed, are either nitrogen donating ligands or metallocene ligands, or a mixture.

As a general rule, EAM comprising non-macrocyclic chelators are bound to metal ions to form non-macrocyclic chelate compounds, since the presence of the metal allows for multiple proligands to bind together to give multiple oxidation states.

In some embodiments, nitrogen donating proligands are used. Suitable nitrogen donating proligands are well known in the art and include, but are not limited to, NH₂; NHR; NRR′; pyridine; pyrazine; isonicotinamide; imidazole; bipyridine and substituted derivatives of bipyridine; terpyridine and substituted derivatives; phenanthrolines, particularly 1,10-phenanthroline (abbreviated phen) and substituted derivatives of phenanthrolines such as 4,7-dimethylphenanthroline and dipyridol[3,2-a:2′,3′-c]phenazine (abbreviated dppz); dipyridophenazine; 1,4,5,8,9,12-hexaazatriphenylene (abbreviated hat); 9,10-phenanthrenequinone diimine (abbreviated phi); 1,4,5,8-tetraazaphenanthrene (abbreviated tap); 1,4,8,11-tetra-azacyclotetradecane (abbreviated cyclam) and isocyanide. Substituted derivatives, including fused derivatives, may also be used. It should be noted that macrocylic ligands that do not coordinatively saturate the metal ion, and which require the addition of another proligand, are considered non-macrocyclic for this purpose. As will be appreciated by those in the art, it is possible to covalent attach a number of “non-macrocyclic” ligands to form a coordinatively saturated compound, but that is lacking a cyclic skeleton.

In some embodiments, a mixture of monodentate (e.g., at least one cyano ligand), bidentate, tri-dentate, and polydentate ligands (till to saturate) can be used in the construction of EAMs

Generally, it is the composition or characteristics of the ligands that determine whether a transition metal complex is solvent accessible. By “solvent accessible transition metal complex” or grammatical equivalents herein is meant a transition metal complex that has at least one, preferably two, and more preferably three, four or more small polar ligands. The actual number of polar ligands will depend on the coordination number (n) of the metal ion. Preferred numbers of polar ligands are (n−1) and (n−2). For example, for hexacoordinate metals, such as Fe, Ru, and Os, solvent accessible transition metal complexes preferably have one to five small polar ligands, with two to five being preferred, and three to five being particularly preferred, depending on the requirement for the other sites, as is more fully described below. Tetracoordinate metals such as Pt and Pd preferably have one, two or three small polar ligands.

It should be understood that “solvent accessible” and “solvent inhibited” are relative terms. That is, at high applied energy, even a solvent accessible transition metal complex may be induced to transfer an electron. The solvent accessible metals and relevant EAMs are described in US Publication Nos. 2011/0033869, 2010/0003710 and 2009/0253149, all of which are expressly incorporated herein in their entirety, and particularly for the figures and definitions outlined therein.

Some examples of EAMs are described herein.

Cyano-Based Complexes

In one aspect, the present invention provides EAMs with a transition metal and at least one cyano (—C≡N) ligand. Depending on the valency of the metal and the configuration of the system (e.g., capture ligand contributing a coordination atom, etc.), 1, 2, 3, 4 or 5 cyano ligands can be used. In general, embodiments which use the most cyano ligands are preferred; again, this depends on the configuration of the system. An EAM using a hexadentate metal such as osmium, separately attached from the capture ligand, allows 5 cyano ligands, with the 6^{th} coordination site being occupied by the terminus of the attachment linker. When a hexadentate metal has both an attachment linker and a capture ligand providing coordination atoms, there can be four cyano ligands.

In some embodiments, the attachment linker and/or the capture ligand can provide more than a single coordination atom. Thus, for example, the attachment linker comprises a bipyridine which contributes two coordination atoms.

In some embodiments, ligands other than cyano ligands are used in combination with at least one cyano ligand.

Ru—N Based Complexes

In one aspect, the resent invention provides new architectures for Ru—N based complexes, where the coordination could be monodentate, bidentate, tridentate, or multidendate. Thus the number of coordination ligand L (which covalently connected to the anchor and capture ligand) can be 1, 2, 3, or 4.

The charge-neutralizing ligands can be any suitable ligand known in the art, such as dithiocarbamate, benzenedithiolate, or Schiff base as described herein. The capture ligand and the anchor can be on the same framework or separate.

In another aspect of the present invention, each component of the EAM ligand architecture is connected through covalent bonds rather than Ru coordination chemistry. The construction of the architectures provide herein relies on modern synthetic organic chemical methodology. An important design consideration includes the necessary orthogonal reactivity of the functional groups present in the anchor and capture ligand component versus the coordinating ligand component.

Preferably, the entire compound can be synthesized and the redox active transitional metal coordinated to the ligand near the last step of the synthesis. The coordinating ligands provided herein rely on well-established inorganic methodologies for ruthenium pentaamine precursors. See Gerhardt and Weck, J. Org. Chem. 71:6336-6341 (2006); Sizova et al., Inorg. Chim. Acta, 357:354-360 (2004); and Scott and Nolan, Eur. J. Inorg. Chem. 1815-1828 (2005), all herein incorporated by reference.

As can be understood by those skilled in the art, the anchor components of the compounds provided herein could be interchanged between alkyl and multipodal-based thiols.

Ferrocene-Based EAMs

In some embodiments, the EAMs comprise substituted ferrocenes. Ferrocene is air-stable. It can be easily substituted with both capture ligand and anchoring group. Upon binding of the target protein to the capture ligand on the ferrocene which will not only change the environment around the ferrocene, but also prevent the cyclopentadienyl rings from spinning, which will change the energy by approximately 4 kJ/mol. WO/1998/57159; Heinze and Schlenker, Eur. J. Inorg. Chem. 2974-2988 (2004); Heinze and Schlenker, Eur. J. Inorg. Chem. 66-71 (2005); and Holleman-Wiberg, Inorganic Chemistry, Academic Press 34^{th} Ed, at 1620, all incorporated by reference.

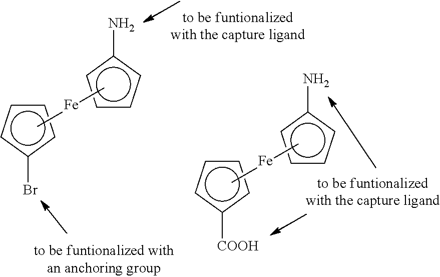

In some embodiments the anchor and capture ligands are attached to the same ligand for easier synthesis. In some embodiments the anchor and capture ligand are attached to different ligands.

There are many ligands that can be used to build the new architecture disclosed herein. They include but not limited to carboxylate, amine, thiolate, phosphine, imidazole, pyridine, bipyridine, terpyridine, tacn (1,4,7-Triazacyclononane), salen (N,N′-bis(salicylidene) ethylenediamine), acacen (N,N′-Ethylenebis(acetylacetoniminate(−)), EDTA (ethylenediamine tetraacetic acid), DTPA (diethylene triamine pentaacetic acid), Cp (cyclopentadienyl), pincer ligands, and scorpionates. In some embodiments, the preferred ligand is pentaamine.

Pincer ligands are a specific type of chelating ligand. A pincer ligand wraps itself around the metal center to create bonds on opposite sides of the metal as well as one in between. The effects pincer ligand chemistry on the metal core electrons is similar to amines, phosphines, and mixed donor ligands. This creates a unique chemical situation where the activity of the metal can be tailored. For example, since there is such a high demand on the sterics of the complex in order to accommodate a pincer ligand, the reactions that the metal can participate in is limited and selective.

Scorpionate ligand refers to a tridentate ligand which would bind to a metal in a fac manner. The most popular class of scorpionates are the tris(pyrazolyl)hydroborates or Tp ligands. A Cp ligand is isolobal to Tp

In some embodiments, the following restraints are desirable: the metal complex should have small polar ligands that allow close contact with the solvent.

Charge-Neutralizing Ligands

In another aspect, the present invention provides compositions having metal complexes comprising charged ligands. The reorganization energy for a system that changes from neutral to charged (e.g., M+<->M0; M−<->M0) may be larger than that for a system in which the charge simply changes (e.g., M2+<->M3+) because the water molecules have to “reorganize” more to accommodate the change to or from an unpolarized environment.

In some embodiments, charged ligand anionic compounds can be used to attach the anchor and the capture ligand to the metal center. A metal complex containing a halide ion X in the inner complex sphere reacts with charged ligands, include but not limited to, thiols (R—SH), thiolates (RS-E; E=leaving group, i.e., trimethylsilyl-group), carbonic acids, dithiols, carbonates, acetylacetonates, salicylates, cysteine, 3-mercapto-2-(mercaptomethyl) propanoic acid. The driving force for this reaction is the formation of HX or EX. If the anionic ligand contains both capture ligand and anchor, one substitution reaction is required, and therefore the metal complex, with which it is reacted, needs to have one halide ligand in the inner sphere. If the anchor and capture ligand are introduced separately the starting material generally needs to contain two halide in the inner coordination sphere. Seidel et al., Inorg. Chem 37:6587-6596 (1998); Kathari and Busch, Inorga. Chem. 8:2276-2280 (1978); Isied and Kuehn J. Am. Chem. Soc. 100:6752-6754; and Volkers et al., Eur. J. Inorg. Chem. 4793-4799 (2006), all herein incorporated by reference.

Examples for suitable metal complexes are the following (it should be noted that the structures depicted below show multiple unidentate ligands, and multidentate ligands can be substituted for or combined with unidentate ligands such as cyano ligands):

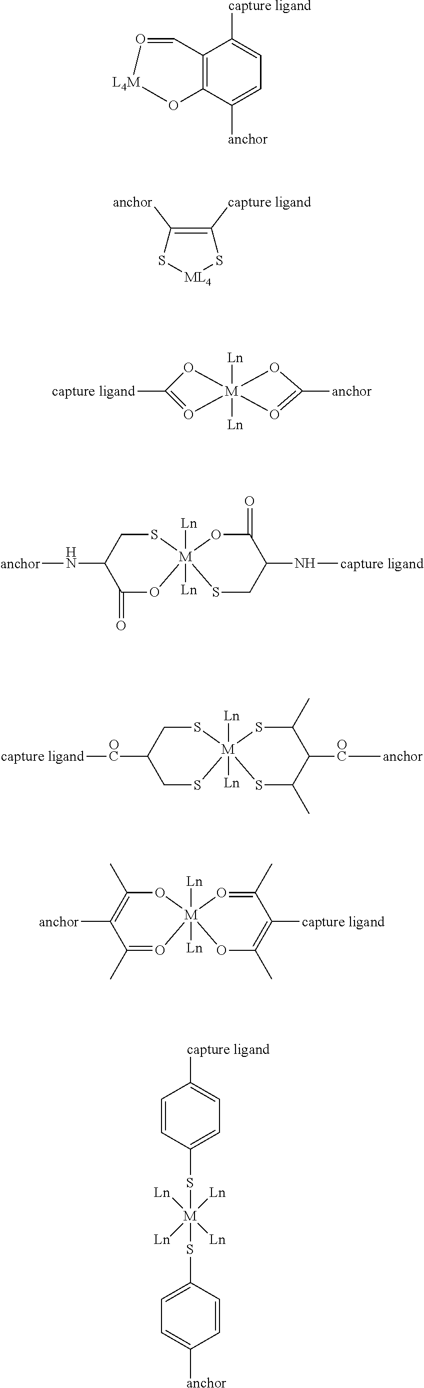

In some embodiments, dithiocarbamate is used as a charge-neutralizing ligand, such as the following example:

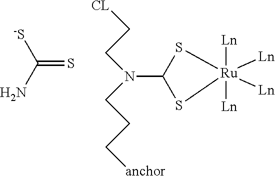

In some embodiments, benzenedithiolate is used as charge-neutralizing ligand, such as the following example:

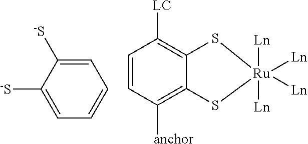

In the above depicted structures, Ln is coordinate ligand and n=0 or 1.

In some embodiments, the EAM comprises Schiff base type complexes. By “Schiff base” or “azomethine” herein is meant a functional group that contains a carbon-nitrogen double bond with the nitrogen atom connected to an aryl or alkyl group—but not hydrogen. Schiff bases are of the general formula R₁R₂C═N—R₃, where R₃ is a phenyl or alkyl group that makes the Schiff base a stable imine Schiff bases can be synthesized from an aromatic amine and a carbonyl compound by nucleophilic addition forming a hemiaminal, followed by a dehydration to generate an imine.

Acacen is a small planar tetradentate ligand that can form hydrogen bonds to surrounding water molecules through its nitrogen and oxygen atoms, which would enhance the reorganization energy effect. It can be modified with many functionalities, include but not limited to, carboxylic acid and halides, which can be used to couple the acacen-ligand to the capture ligand and to the anchoring group. This system allows a large variety of different metal centers to be utilized in the EAMs. Since the ligand binds with its two oxygen and two nitrogen atoms, only four coordination sites are occupied. This leaves two additional coordination sites open, depending on the metal center. These coordination sites can be occupied by a large variety of organic and inorganic ligands. These additional open sites can be used for inner-sphere substitution (e.g., labile H₂O or NH₃ can be displaced by protein binding) or outer-sphere influence (e.g., CO, CN can for H-bonds) to optimize the shift of potentials upon binding of the capture ligand to the target. WO/1998/057158, WO/1997/21431, Louie et al., PNAS 95:6663-6668 (1999), and Bottcher et al., Inorg. Chem. 36:2498-2504 (1997), herein all incorporated by references.

In some embodiments, salen-complexes are used as well. Syamal et al., Reactive and Functional Polymers 39:27-35 (1999).

The structures of some acacen-based complexes and salen-based complexes are shown below, where positions on the ligand that are suitable for functionalization with the capture ligand and/or the anchor are marked with an asterisk.

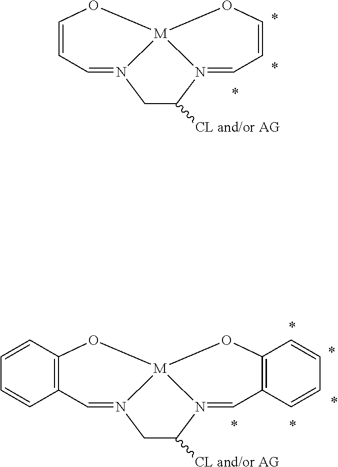

One example of using acacen as ligand to form a cobalt complex is the following:

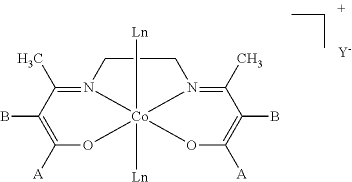

wherein is A and B are substitute groups, Ln is coordinating ligand and n=0 or 1.

Sulfato Ligands

In some embodiments, the EAM comprises sulfato complexes, include but not limited to, [L-Ru(III)(NH₃)₄SO₄]⁺ and [L-Ru(III)(NH₃)₄SO₂]2⁺. The SO₄—Ru(III)-complexes are air stable. The ligand L comprises a capture ligand an anchor. The sulfate ligand is more polar than amine and negatively charged. The surface complexes therefore will be surrounded by a large number of water molecules than both the [L-Ru(NH₃)₅-L′] and [L-Ru(NH₃)₅]2⁺. Isied and Taube, Inorg. Chem. 13:1545-1551 (1974), herein incorporated by reference.

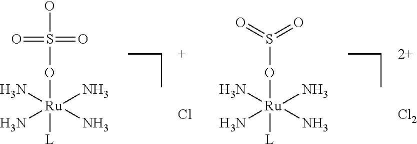

Spacer Groups

In some embodiments, the EAM or ReAMC is covalently attached to the anchor group (which is attached to the electrode) via an attachment linker or spacer (“Spacer 1”), that further generally includes a functional moiety that allows the association of the attachment linker to the electrode. See for example U.S. Pat. No. 7,384,749, incorporated herein by reference in its entirety and specifically for the discussion of attachment linkers). It should be noted in the case of a gold electrode, a sulfur atom can be used as the functional group (this attachment is considered covalent for the purposes of this invention). By “spacer” or “attachment linker” herein is meant a moiety which holds the redox active complex off the surface of the electrode. In some embodiments, the spacer is a conductive oligomer as outlined herein, although suitable spacer moieties include passivation agents and insulators as outlined below. In some cases, the spacer molecules are SAM forming species. The spacer moieties may be substantially non-conductive, although preferably (but not required) is that the electron coupling between the redox active molecule and the electrode (HAB) does not become the rate limiting step in electron transfer.

In addition, attachment linkers can be used to between the coordination atom of the capture ligand and the capture ligand itself, in the case when ReAMCs are utilized. Similarly, attachment linkers can be branched. In addition, attachment linkers can be used to attach capture ligands to the electrode when they are not associated in a ReAMC.

One end of the attachment linker is linked to the EAM/ReAMC/capture ligand, and the other end (although as will be appreciated by those in the art, it need not be the exact terminus for either) is attached to the electrode.

The covalent attachment of the conductive oligomer containing the redox active molecule (and the attachment of other spacer molecules) may be accomplished in a variety of ways, depending on the electrode and the conductive oligomer used. See for example Structures 12-19 and the accompanying text in U.S. Patent Publication No. 20020009810, hereby incorporated by reference in its entirety.

In general, the length of the spacer is as outlined for conductive polymers and passivation agents in U.S. Pat. Nos. 6,013,459, 6,013,170, and 6,248,229, as well as U.S. Pat. No. 7,384,749 all herein incorporated by reference in their entireties. As will be appreciated by those in the art, if the spacer becomes too long, the electronic coupling between the redox active molecule and the electrode will decrease rapidly.

Method of Making

In another aspect, the present invention provides method of making the compositions as described herein. In some embodiments, the composition are made according to methods disclosed in of U.S. Pat. Nos. 6,013,459, 6,248,229, 7,018,523, 7,267,939, U.S. patent application Ser. Nos. 09/096,593 and 60/980,733, and U.S. Provisional Application No. 61/087,102, filed on Aug. 7, 2008, all are herein incorporated in their entireties for all purposes.

In one embodiments, Compound 1 (an unsymmetric dialkyl disulfide bearing terminal ferrocene and maleimide groups) as shown below was synthesized and deposited on gold electrodes as previously described.

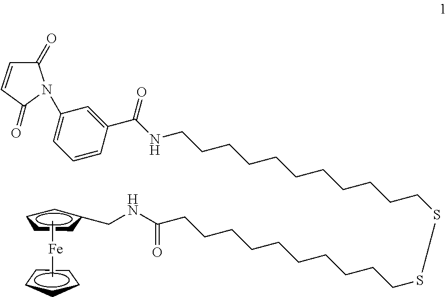

Diagnosis and Prognosis

The present invention provides for the diagnosis and prognosis of prostatic disease based on enzymatic activity against a PCSP in a sample, and in particular, the enzymatic activity of PSA in the sample.

The present invention also provides for the prognosis of prostate cancer based on the same assay of enzymatic activity against a PCSP in a sample, and in particular, the enzymatic activity of PSA in the sample. This is in part accomplished by evaluating the aggressiveness of prostate cancer through the same assay of enzymatic activity against a PCSP in a sample, and in particular, the enzymatic activity of PSA in a sample.

In some embodiments, Receiver Operating Characteristic (ROC) curve analysis is done to assess the sensitivity and specificity of a chosen biomarker at different cut-off points. Each point on the ROC curve represents a sensitivity/specificity pair corresponding to a particular decision threshold for the value of the biomarker (normalized or not) as chosen. As is known in the art, ROC curves are a fundamental tool for diagnostic or prognostic test evaluation. In a ROC curve the true positive rate (Sensitivity) is plotted in function of the false positive rate (100-Specificity) for different cut-off points of a parameter or parameters. Each point on the ROC curve represents a sensitivity/specificity pair corresponding to a particular decision threshold. The area under the ROC curve is a measure of how well a parameter can distinguish between two diagnostic groups (diseased/normal) or two prognostic groups (aggressive/non-aggressive. Thus, ROC curve analysis is done to evaluate the diagnostic performance of a test, or the accuracy of a test to discriminate diseased cases from normal cases (Metz, 1978; Zweig and Campbell, 1993). ROC curves can also be used to compare the diagnostic performance of two or more laboratory or diagnostic tests (Griner et al., 1981).

In the present invention, ROC curves are generated in a blind study using one or a combination of parameters as discussed below with established samples, e.g., preconfirmed (independent diagnosis) samples which classifies the previous subjects into two distinct groups: a diseased and non-diseased group.

In the present invention, ROC curves are generated using a single parameter, e.g., enzymatic activity against a PCSP or PSA enzymatic activity in a sample as defined herein.

Alternatively, ROC curves are generated using one or more parameters optionally and independently selected from the list including, but not limited to, a) enzymatic activity in the sample; b) prostate volume; c) Gleason score; c) total, free and or ratio of f/tPSA in serum; d) total PSA in the sample tested for activity; f) volume of prostatic fluid (generally normalized using zinc concentration as is known in the art); g) amount of urine (generally normalized using creatininine amount); h) HGPin and i) PIN.

In some embodiments, the enzymatic activity and any other parameter in the above list can be combined. In some embodiments, two parameters are used to generate the ROC curves, including, but not limited to, a) enzymatic activity in the sample and prostate volume; b) enzymatic activity in the sample and total PSA (including active and non-active (e.g. bound) in the sample; c) enzymatic activity in the sample and total PSA (including active and non-active (e.g. bound) in the serum of the patient; d) enzymatic activity in the sample and Gleason score.

In some embodiments, three parameters are used to generate the ROC curves, including, but not limited to, a) enzymatic activity in the sample, amount of total PSA in the sample and prostate volume, and b) enzymatic activity in the sample, amount of total PSA in the serum and prostate volume.

As will be appreciated by those in the art, the multiparameter analysis can be done by division (e.g. enzymatic activity in the sample divided by prostate volume) or multiplication, or any other way of forming a constant.

Once generated, a specific value can be obtained which allows for diagnosis of new clinical samples when compared to the threshold established by the ROC curves.

Additionally or alternatively, the single or multiparameter analyses can be integrated into existing prostate cancer and prostate disease risk nomograms. As is well known in the art, nomograms are generated using a variety of factors, to which the enzymatic activity against a PCSP and/or PSA enzymatic activity from a sample can be added.

Optionally or additionally, ROC curves can be generated using samples from two or more of normal (e.g., free of disease) patients, prostate cancer patients, and/or non-cancer prostatic disease (e.g., BPH) patients as well as from two patient populations of aggressive and non-aggressive. These ROC curves can be generated using enzymatic activity in a sample normalized to one or more of the following factors: a) prostate volume; b) Gleason score; c) total, free and or ratio of free/total PSA in serum; d) total PSA in the sample tested for activity; e) volume of prostatic fluid (generally normalized using zinc concentration as is known in the art); f) amount of urine (generally normalized using creatin levels); g) HGPin and h) PIN.

In an alternative embodiment, zymography is used to determine the enzymatic activity of the protease(s) in the sample against a PCSP. Zymography is an electrophoretic technique wherein the sample is generally run under native conditions (e.g., in the absence of reducing agents and detergents) either in a gel that contains a substrate or using a post-electrophoretic gel overlay. As noted by Webber et al., PSA has shown gelatinolytic protease activity by PSA-SDS-PAGE zymography, a method used to evaluate the extracellular matrix degrading ability of a protease. Webber et al. describe the measurement of PSA activity using the degradation of fibronectin and laminin per the proteases physiological activity against semenogelin and fibronectin in semen. Webber et al., (1995) Clin. Cancer Res. 1:1089, incorporated by reference. Thus, in one embodiment, the substrate is incorporated into the gel, which can be either a fibronectin-like substrate, with measurements generally based on the alteration of the opacity of the gel where the enzyme is, or on the generation of a chromogenic signal based on the use of optical peptide substrates as outlined herein. As an alternative to incorporating the substrate in the gel, overlay gels can be used at the conclusion of the electrophoretic run, with either an additional gel or a solution containing the chromogenic substrate being added to the gel. In general, calibration is done either with a densitometer or with an optical reader (including fluorimeters, when the substrate is fluorogenic).

The role of prostate specific antigen (PSA) in prostate cancer is not clear. Although used as a biomarker for prostate cancer, the correlation with cancer is not necessarily straightforward. The present invention provides a simple assay correlated with the presence or absence of prostate cancer, with an ability to distinguish between aggressive and non-aggressive disease.

This method is a non-invasive test for the aggressiveness of prostate cancer. The success of such a test would eliminate over diagnosis and prevent the side effects of the ensuing treatment. Many patients diagnosed with low risk cancer continue to opt for treatment or radical surgery⁶. An accurate test for aggressive versus indolent forms of cancer could greatly relieve the physical and financial stress, among other burdens put on patients who undergo surgery.

### EXAMPLES

### Example 1

778 prostatic fluid samples were collected in the operating room from post-radical prostatectomy (PRP) specimens immediately after resection. Specimens were frozen at −80° C. Biochemical, clinical and surgical pathology information was obtained before and after surgery and recorded in an IRB-approved prospective research database. 50 samples from clinically aggressive and 50 from clinically non-aggressive cases were randomly selected for use in this unmatched case-control pilot study. Clinically aggressive prostate cancer was defined as cancer resulting in prostate cancer-specific death, lymph node or distant metastases, seminal vesicle invasion, or extracapsular tumor extension. Clinically non-aggressive prostate cancer was defined as cancer with Gleason score <6, pathology stage T2, and no evidence of clinical or biochemical tumor recurrence. By these definitions, 25.4% and 32.8% of subjects had clinically aggressive or non-aggressive prostate cancer respectively. The remaining 41.8% had prostate cancer that fell somewhere in the middle of the “aggressiveness spectrum.” Since this pilot study was intended to enable an initial determination between the most and least aggressive prostate cancer the specimens chosen were from the tails of the aggressiveness distribution of the overall population.

Materials

PSA substrate HSSKLQ-AMC (AMC=4-methyl-coumaryl-7-amide) was purchase from Peptides International (Louisville, Ky.) and PSA was purchased from Scripps Laboratories (San Diego, Calif.). Stock solutions of HSSKLQ-AMC (0.4 mM) were made in buffer A (50 mM Tris-HCl, 1.5M NaCl, 2 mg/mL BSA, pH=7.5). The PRP prostatic fluid samples were obtained from Northwestern University Pathology Core under a materials transfer agreement that was exempt approved by the Northwestern University IRB. Each prostatic fluid sample (50 μL) was thawed, gently mixed, and aliquotted into ten equal aliquots and stored at −80° C.

Enzymatic Activity Assay

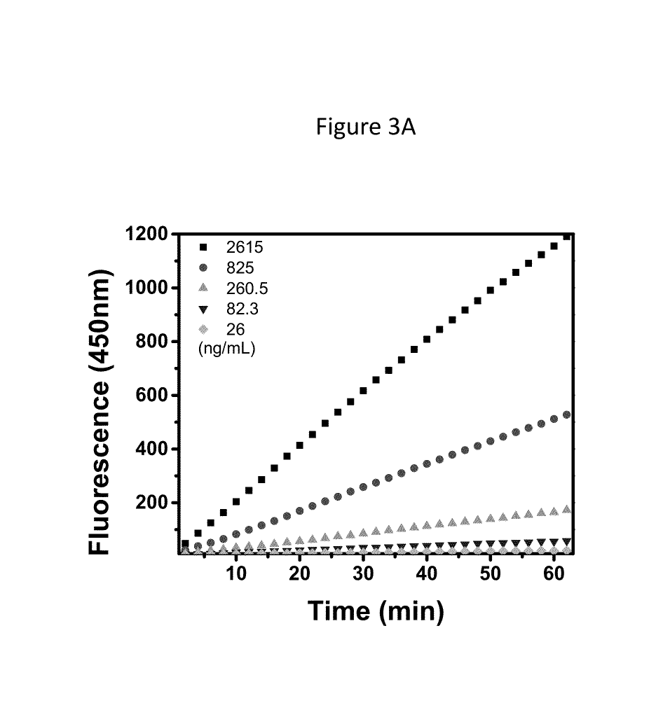

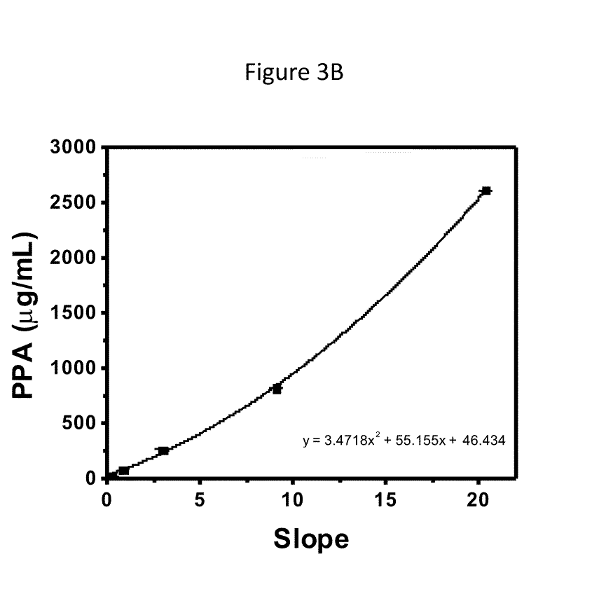

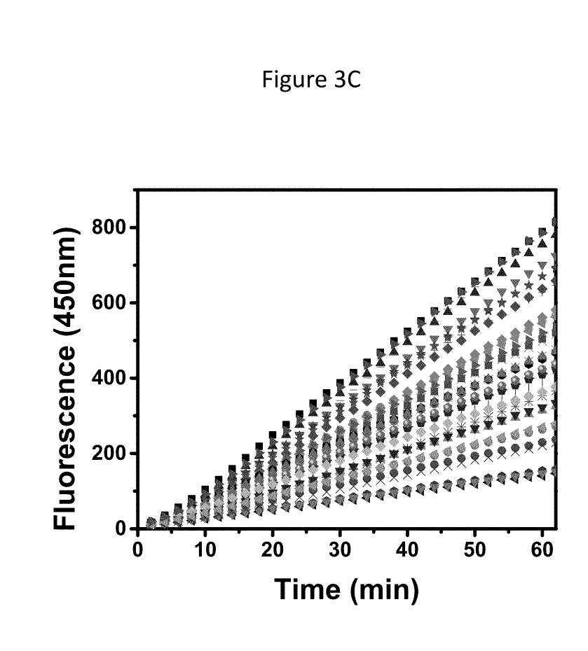

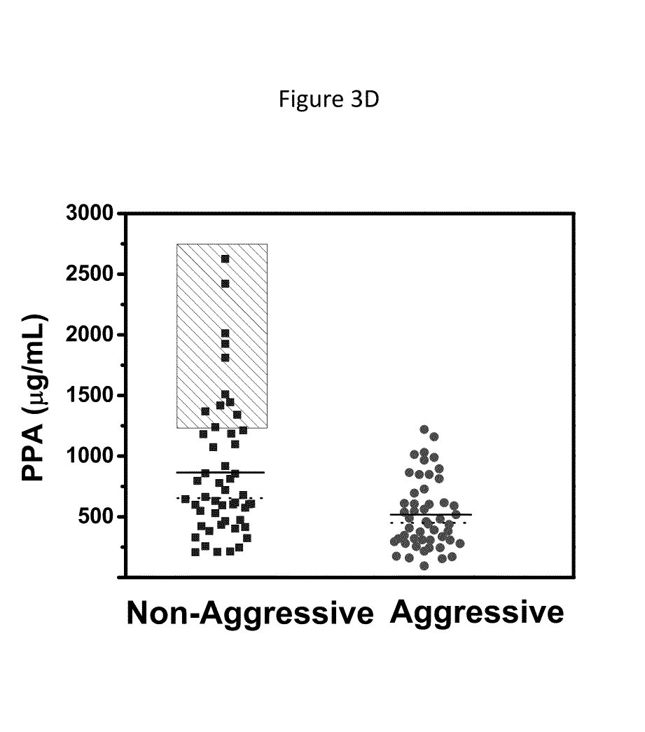

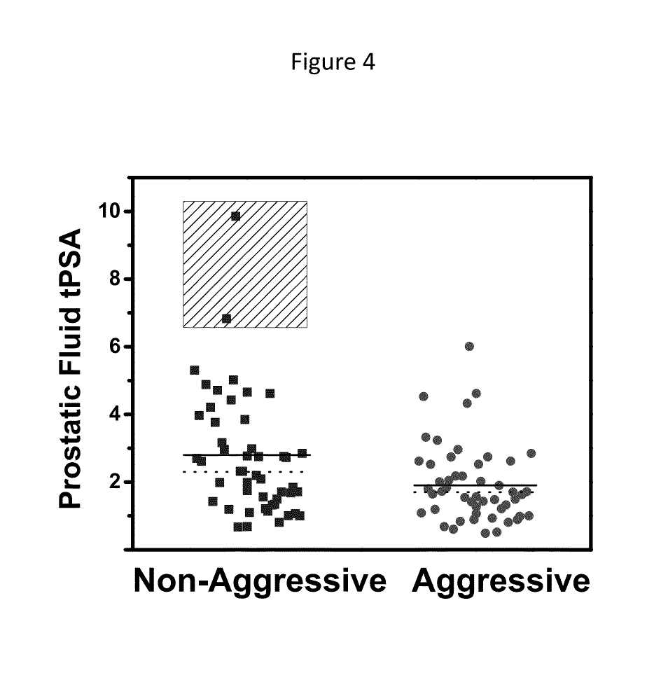

The prostatic fluid samples (5 μL) were diluted in 70 μL of buffer A followed by vigorous mixing. The diluted samples (2 μL) were loaded in triplicate onto a 96-well microplate followed by 98 μL of peptide solution (0.4 mM HSSKLQ-AMC in buffer A). A serial dilution of PSA (2615 to 26 ng/mL) in buffer A was run concurrently with the samples to generate a standard curve (see FIG. 3A, 3B). All prostatic fluid samples with standard curves were run a total of three times. All reactions were run on a Biotek Synergy 4 microplate reader operating in top read fluorescence mode (380 nm excitation/450 nm emission) at room temperature for 60 minutes reading every 2 minutes.

Data Analysis

The fluorescence intensity data collected in triplicate was averaged, plotted as fluorescence versus time (see FIG. 3C), and the slope fit by linear regression analysis. A standard curve was generated by plotting the PSA concentration versus slope and fitting with a bi-exponential curve (see FIG. 3B; r²≧0.999). The measured slope for each sample was input into the standard curve and the enzymatic activity of PSA was calculated. All statistical analyses were performed using SAS® 9.2.

Results

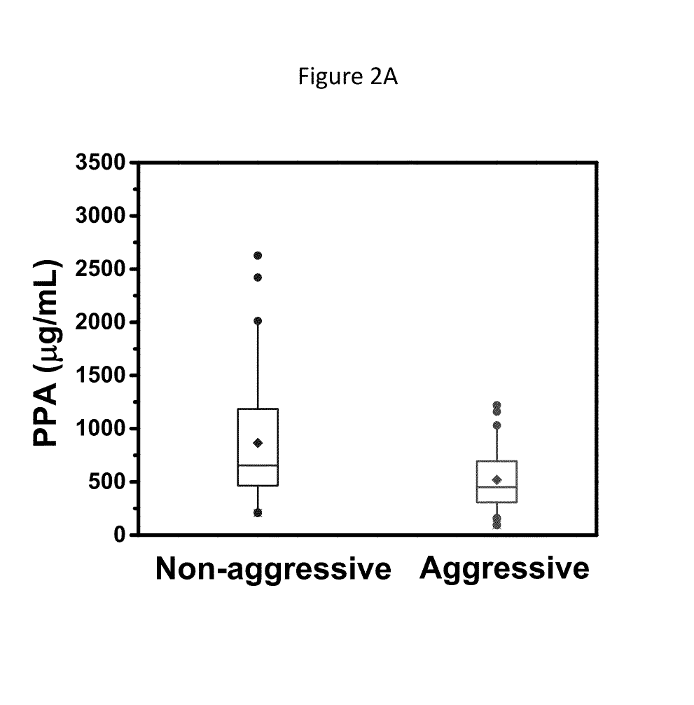

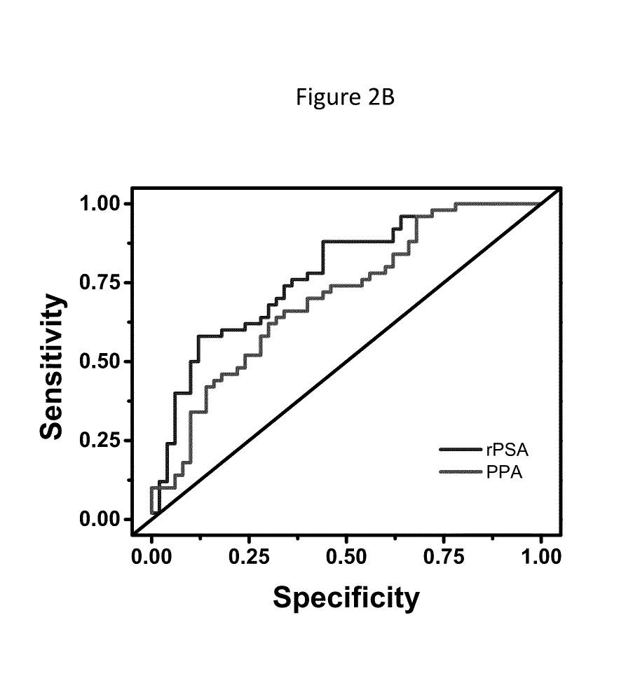

In this blinded unmatched case-control study, the enzymatic activity of PSA for each prostatic fluid sample was measured using the protocols described as described above, and the data were de-identified and sorted (see FIGS. 2A & 3D). The clinically non-aggressive population was observed to have a significantly higher enzymatic activity of PSA value (mean=865 μg/mL; median=654 μg/mL) than the clinically aggressive population (mean=518 μg/mL; median=449 μg/mL). Thus, there was a negative association of enzymatic activity of PSA with cancer aggressiveness. A ROC analysis appropriate for an unmatched case-control study was then performed to assess the highest diagnostic effect for predicting aggressive prostate cancer (see FIG. 2B). Among factors considered (including age, prostate weight and serum total PSA (tPSA)), prostatic fluid enzymatic activity of PSA and the normalized ratio of prostatic fluid enzymatic activity of PSA/serum tPSA (rPSA) had the highest discriminatory power for predicting the presence of aggressive prostate cancer. An area under the curve (AUC) was calculated: 0.7008 [95% CI: (0.5986, 0.8030)] for enzymatic activity of PSA and 0.7784 [95% CI: (0.6880, 0.8688)] for rPSA with the latter being significantly higher (p-value=0.0300 based on a Chi-square test; see Delong, E. R et al, (1988) Biometrics 44, 837-845). Based on the ROC analyses, two separate logistic regression analyses for an unmatched case-control study were performed to estimate the extent to which prostate cancer pathology was associated with enzymatic activity of PSA (adjusted for age, serum tPSA, and prostate weight) and rPSA (adjusted for age and prostate weight). The results showed that enzymatic activity of PSA and rPSA each had a significant inversely proportional association with prostate cancer pathology [enzymatic activity level of PSA: odds ratio=0.799 per 100 ug/mL increase, 95% CI=(0.678, 0.942), p-value=0.0074; rPSA: odds ratio=0.884 per 10 ug/ng increase, 95% CI=(0.827, 0.945), p-value=0.0003].

### Example 2

### Example 2.A

### Optical Assay for Measurement of PSA Enzymatic Activity

Reagents

Buffer A: 50 mM Tris-HCl, 1.5 M NaCl, 2 mg/mL BSA, pH=7.5, Mor-HSSKLQ-AMC; Peptides Int.; MW=956.03 g/mol; 0.4 mM in buffer A. Mor-HSSK-Hic-Q-AMC; Peptides Int.; MW=970.06 g/mol; 0.4 mM in buffer A. Mor-HSSK-Hiv-Q-AMC; Peptides Int.; MW=955.44 g/mol; 0.4 mM in buffer A. PSA; Scripps Laboratories, 1 aliquot (2 ug/20 μL); MW=33,000; add 586 μL buffer A (=100 nM). 7-Amino-4-methylcoumarin (AMC); Aldrich, MW=175.18 g/mol, 22.2 mM in DMSO. Anticatalytic mAb M0750; Dako; 66 mg/mL, α-Chymotrypsin; Sigma (C3142); MW=25,000; 100 nM in buffer A, Trypsin—Type 1; Sigma (T8003); MW=23,800; 100 nM in buffer A, tosylphenylalanine chloromethylketone (TPCK); Acros, 99%, MW=351.84 g/mol, 21 mM in DMSO, Phenylmethanesulfonyl fluoride (PMSF); Sigma, 98.5%, 174.19 g/mol, 21 mM in DMSO, ZnCl₂; Aldrich, 136.3 g/mol; 220 nm in buffer A (without BSA).

Samples: D1-D47 clinical urine samples (500 μL each) were divided into 10 aliquots and stored at −80° C. until use. Male urine control from anonymous lab volunteer. Female urine control from anonymous lab volunteer.

Equipment: Biotek Synergy™ 4 multiplate reader; fluorescence mode (380 nm excit./450 nm emiss.); Costar 96-well microplates (Corning, #3603)

Experimental Outline

Serial dilution of AMC (reagent #6) to determine linear fluorescence range

Serial dilution of PSA (reagent #5)+substrate

Mor-HSSKLQ-AMC (reagent #2)

Mor-HSSK-Hic-Q-AMC (reagent #3)

Mor-HSSK-Hiv-Q-AMC (reagent #4)

Serial dilution of α-Chymotrypsin (reagent #8)+substrate

Mor-HSSKLQ-AMC (reagent #2)

Mor-HSSK-Hic-Q-AMC (reagent #3)

Mor-HSSK-Hiv-Q-AMC (reagent #4)

Serial dilution of Trypsin—Type 1 (reagent #9)+substrate

Mor-HSSKLQ-AMC (reagent #2)

Mor-HSSK-Hic-Q-AMC (reagent #3)

Mor-HSSK-Hiv-Q-AMC (reagent #4)

Inhibition of PSA and Chymotrypsin activity with TPCK (reagent #10) and PMSF (reagent #11)

a. Mor-HSSKLQ-AMC (reagent #2) as substrate

b. Mor-HSSK-Hiv-Q-AMC (reagent #4) as substrate

D1-D47 clinical samples (duplicate; 50 μL+150 μL substrate #2)

D1-D47 clinical samples (singlet; 50 μL+150 μL substrate #2)

D1-D47 clinical samples (singlet; 50 μL+150 μL substrate #2)+neat clinical samples.

D5, D6, D21, D22, D27, D29, clinical samples (singlet; 50 μL+substrate #3)+neat samples.

Anticatalytic activity mAb+substrate (reagent #2)+D39 (or D40)

General Procedure for Microplate Experiment

The desired clinical urine samples were thawed at room temperature, gently vortexed, and briefly centrifuged (<20 seconds) to accumulate sample at the bottom of the tube. Each 50 μL sample was transferred via pipette to the 96-well microplate. The PSA standards (20 μL) were prepared and loaded in the same way. A multichannel pipette was used to transfer the substrate (150 μL) one column at a time and the start time recorded. Once the entire plate was loaded, it was inserted into the microplate reader and analyzed every 10 min. for 2-12 hrs.

General Procedure for Protein Dilution

A series of 7 low-bind microcentrifuge tubes were arranged and 190 μL of protein stock solution added to tube #1 and 130 μL buffer A added to the remaining tubes. Transferred 60 μL from tube 1 to 2, vortexed and briefly centrifuged. Removed 60 μL from tube 2 and added to tube 3; vortexed, centrifuged. This gave a final concentration range of 25.0 nM-0.25 nM.

Experimental Details

Serial dilution of AMC (reagent #6) to determine linear fluorescence range: Reagent #6 (35.9 μL) was diluted to 2.0 mL buffer A to give a concentration of 0.4 mM. A 1:2 dilution was performed to give a final concentration range of 0.4 mM-0.024 uM. This was loaded into a 96-well microplate in duplicate and scanned one time. Serial dilution of PSA (reagent #5)+substrate: 20 μL of each standard PSA standard solution (see general procedure for protein dilution above) was loaded in duplicate into a 96-well microplate followed by 150 μL of peptide substrate in buffer A (see general procedure above) and scanned for at least 3 hrs. Serial dilution of α-Chymotrypsin (reagent #8)+substrate: Same as experiment 2 but with reagent #8. Serial dilution of Trypsin—Type 1 (reagent #9)+substrate: Same as experiment 2 but with reagent #9.

Inhibition of PSA and Chymotrypsin activity with TPCK, PMSF, and zinc. A 96-well microplate was loaded with 20 μL of enzyme solution (133.3 nM in buffer A; see plate map below). 190 μL of reagents #2 & #4 were loaded into columns 8-12. Using a multichannel pipette, 180 μL of each substrate solution was transferred to begin the reaction (7 to 1; 8 to 2; 9 to 3; 10 to 4; 11 to 5; 12 to 6). The plate was read for 77 min, scanning every 10 min. then 10 μL of the respective inhibitor added to the corresponding wells. The plate was read for another 123 minutes.

D1-D47 Clinical Samples (Duplicate; 50 μL+150 μL Substrate #2)

Clinical samples D1-D47 were loaded into a 96-well microplate (see procedures above) along with a standard dilution series of PSA (in duplicate). 150 μL of reagent #2 was added to each column to begin the reaction and the plate scanned every 10 min for 12 hrs.

D1-D47 Clinical Samples (Singlet; 50 μL+150 μL Substrate #2)

Clinical samples D1-D47 were loaded into a 96-well microplate (see procedures above) along with a standard dilution series of PSA (in duplicate). 150 μL of reagent #2 was added to each column to begin the reaction and the plate scanned every 10 min for 12 hrs.

D1-D47 Clinical Samples (Singlet; 50 μL+150 μL Substrate #2)+Neat Clinical Samples.

Clinical samples D1-D47 were loaded in duplicate into a 96-well microplate (see procedures above) along with a standard dilution series of PSA (in duplicate). 150 μL of reagent #2 was added to the first set of clinical samples and to each column of the PSA dilution series. The other set of clinical samples were diluted with 150 μL of buffer A (to enable subtraction of urine autofluorescence). The plate was scanned every 10 min for 8 hrs.

D5, D6, D21, D22, D27, D29 Clinical Samples (Singlet; 50 μL+Substrate #3)+Neat Samples.

Clinical samples were loaded into a 96-well microplate in duplicate. Reagent #3 (150 μL) was added to the first set will 150 μL of buffer A was added to the second set. The plate was read every 10 min. for 4 hrs.

Anticatalytic Activity mAb+Substrate (Reagent #2)+D39 (or D40)

Data Analysis: Data obtained from samples that were run in neat buffer were plotted as fluorescence versus time. Samples (clinical or control) that were run in urine were run side-by-side with the neat urine sample (without substrate) and the background autofluorescence subtracted from the sample+substrate data. This was then plotted as fluorescence versus time.

To measure the slope (activity), the data from time 100 min to 200 min was subjected to linear regression analysis and the slope obtained from the best-fit line. Any data with an R2 value of less than 0.9 was set aside and examined on a case-by-case basis.

### Example 2.B

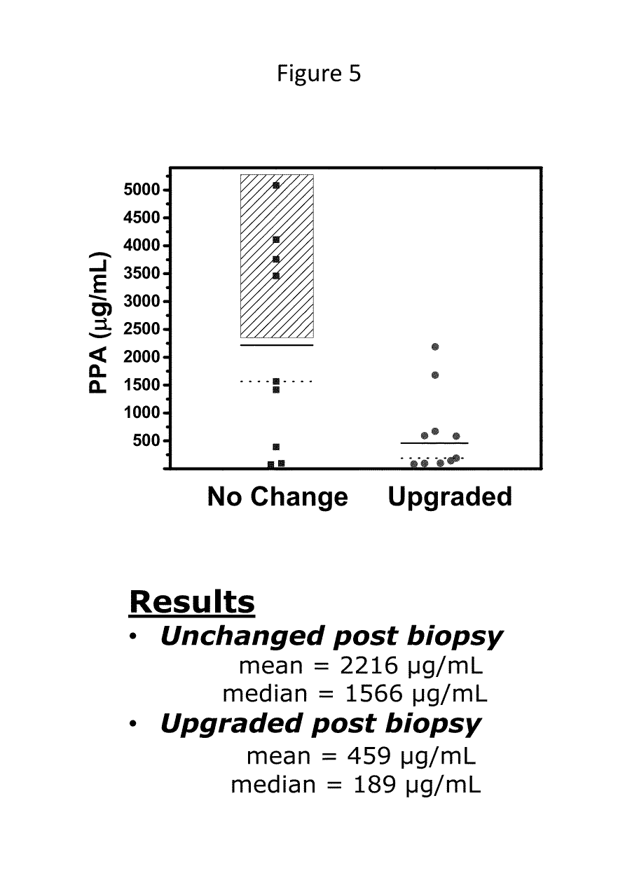

20 out 646 expressed prostatic fluid were obtained by the City of Hope Medical Center and were analyzed by measuring enzymatic activity levels of PSA and analyzed as described above in Example 2.A. The projected result was that patients diagnosed with aggressive prostate cancer would have low enzymatic activity levels of PSA and the inverse for patients with non-aggressive cancer. The 20 samples were correlated with many different variants. Classification of non-aggressive (NA), intermediate (INT), and aggressive (A) cancer was based on a prior study done by Ohmx (see Ahrens, M. J. et al. (2013) The Prostate DOI 10. 1002/pros.22714) in accordance with NCCN guidelines (see “NCCN Guidelines for Patients®|Prostate Cancer.” National Comprehensive Cancer Network). The differences between clinical diagnosis and later pathological examinations seemed to have a trend of upgrading the severity of the cancers. The enzymatic activity levels of PSA were normalized with reference to PSA levels in serum and prostatic fluid volumes. The aggressive cancers have generally low levels, and that the proteolytic activity of PSA helps predict any cancer upstaging and upgrading between clinically determined values and surgically determined values. Results are summarized in FIG. 5.

### Example 3

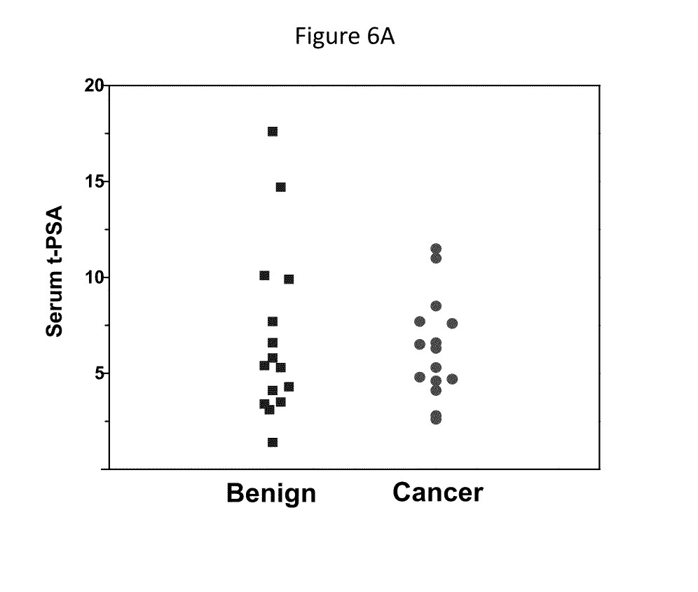

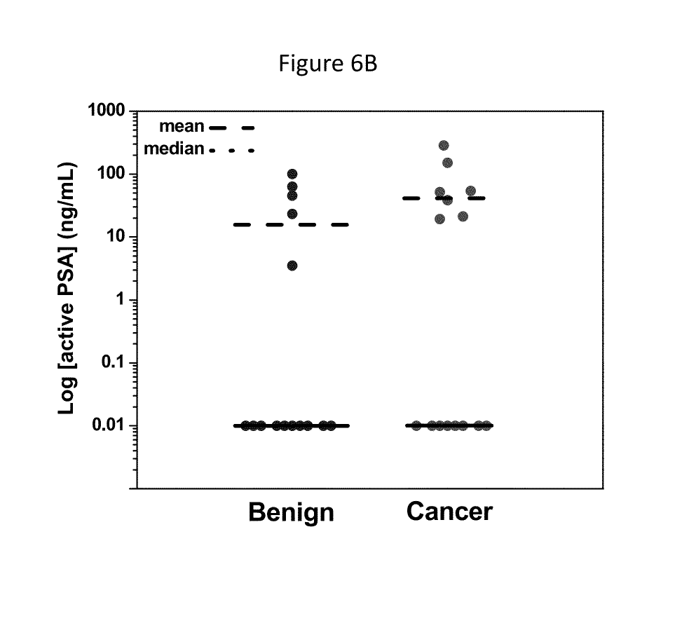

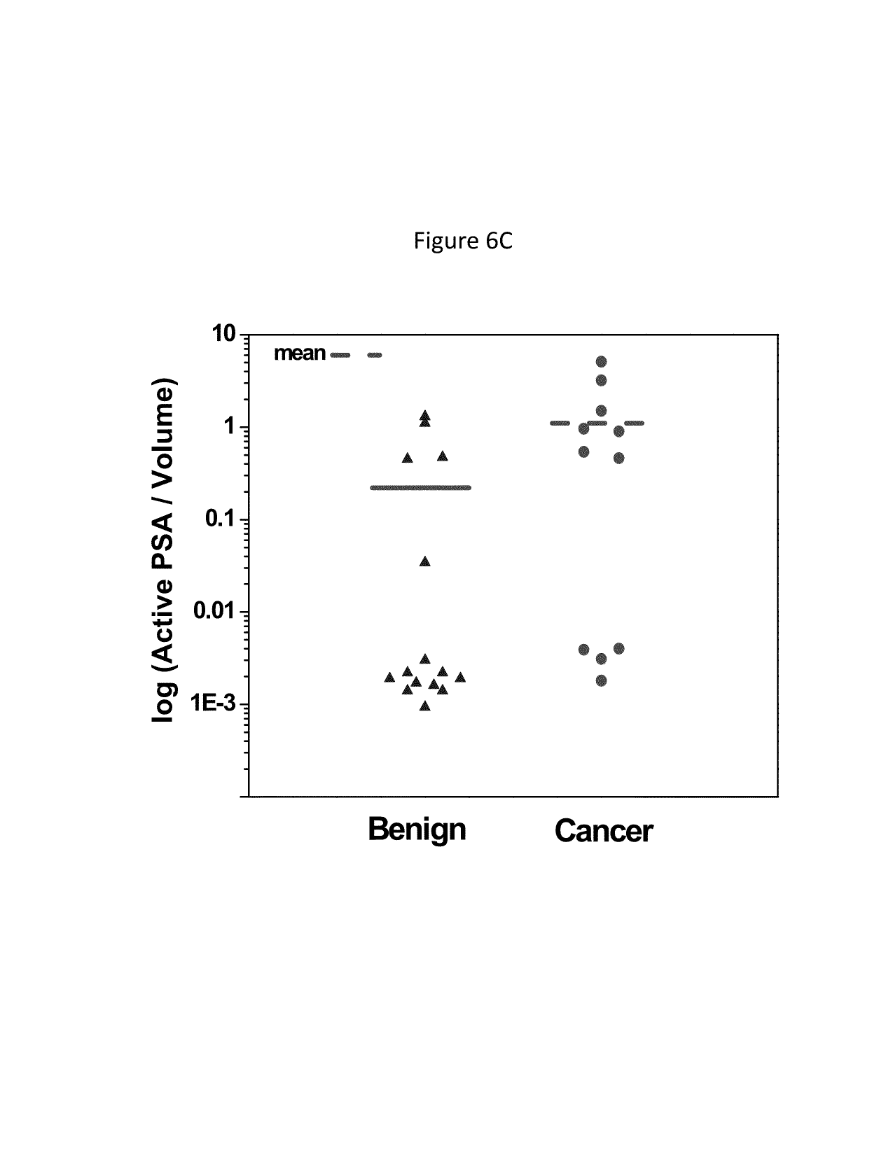

30 clinical urine samples were obtained from the Urological Research Foundation and analyzed as described above in Example 2.A. The de-identified urine samples were collected following a DRE prostatic massage from patients with elevated serum tPSA. The samples included 15 positive biopsy-confirmed prostate cancer patients with Gleason scores of 6 or greater and 15 negative patients with normal prostate biopsies but with BPH. Using the commercially obtained fluorogenic peptide HSSKLQ-AMC, the fluorescence cleavage assay was blindly performed as described previously. Denmeade et al. (1997) Can Res. 57:4924-4930. The results are shown in FIG. 6. The majority of negative control samples showed minimal PSA activity, in contrast to the high median PSA activity levels from the cancer-confirmed group, which is total opposite to the results for serum t-PSA levels. An extended statistical analysis was done to assert whether there are other values that can contribute to this activity.

It was identified that the prostate volume of patients contributes to the false positives and false negatives. Accordingly, the activity data was normalized for prostate volume (e.g., peptide activity over patient prostate volume), resulting in statistically different values for the two populations. Additionally, a similarly better correlation was also established with the normalization of activity of amount of total PSA in the urine samples.

### Example 4

Another set of 47 post-DRE urine samples were collected and analyzed as described above in Example 2.A. The same PSA proteolytic activity is identified as before. For this set the samples on their own were also tested and the autofluorescence of urine was subtracted from the activity curves and better results were obtained. This step was not run in the prior study performed with 30 samples.

For these data again the commercial serum t-PSA value not only does not show any correlation, but it actually is a negative biomarker. As opposed to what would be expected the mean t-PSA for cancer patients is lower than the mean t-PSA for BPH patients. For the PSA activity however, the mean for cancer patients is higher than the mean of BPH patients, consistent with the findings of previous studies in the literature.

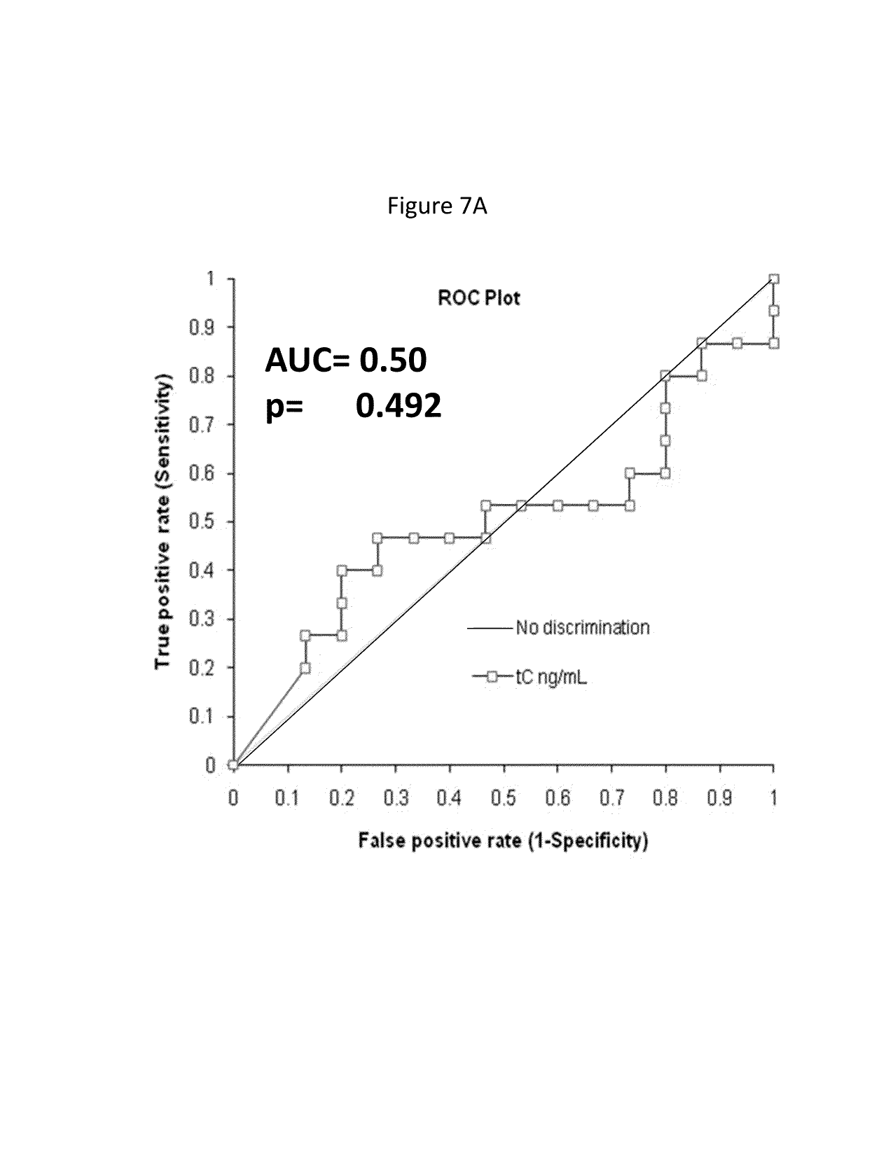

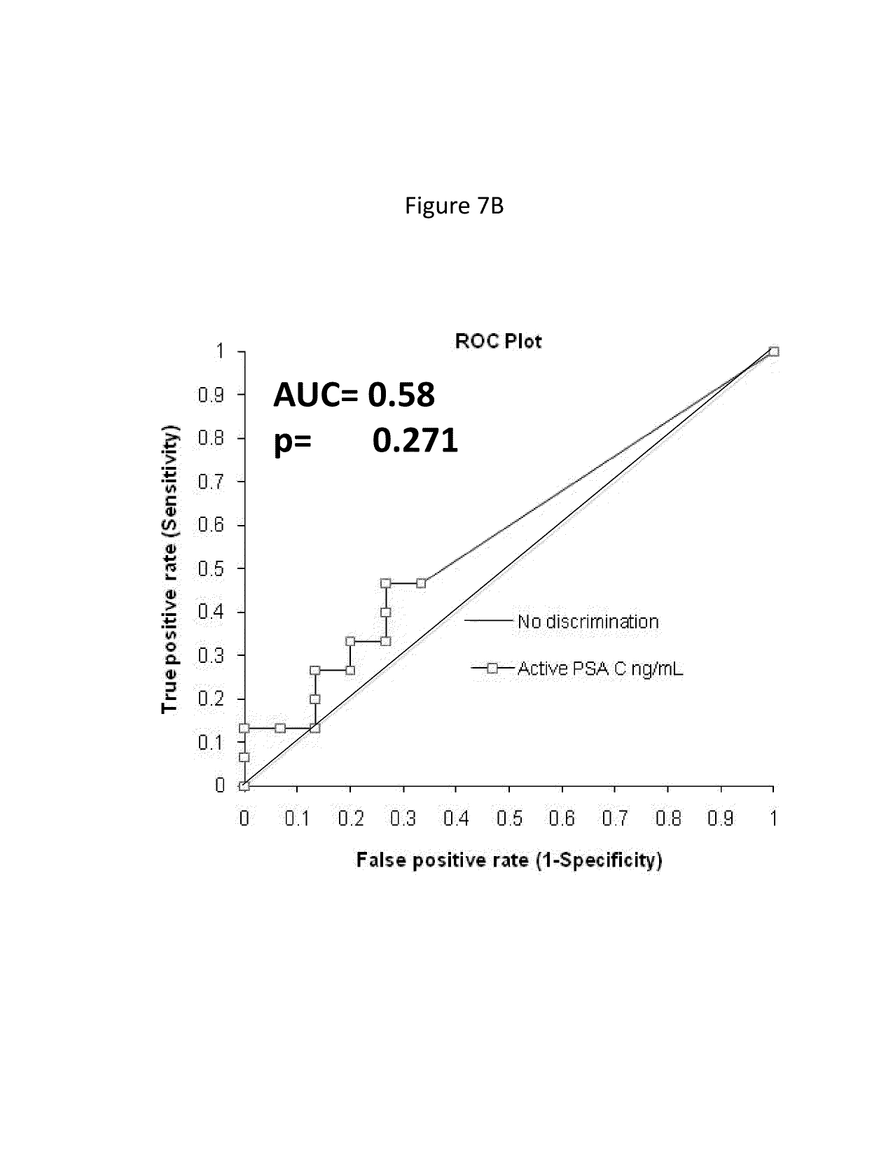

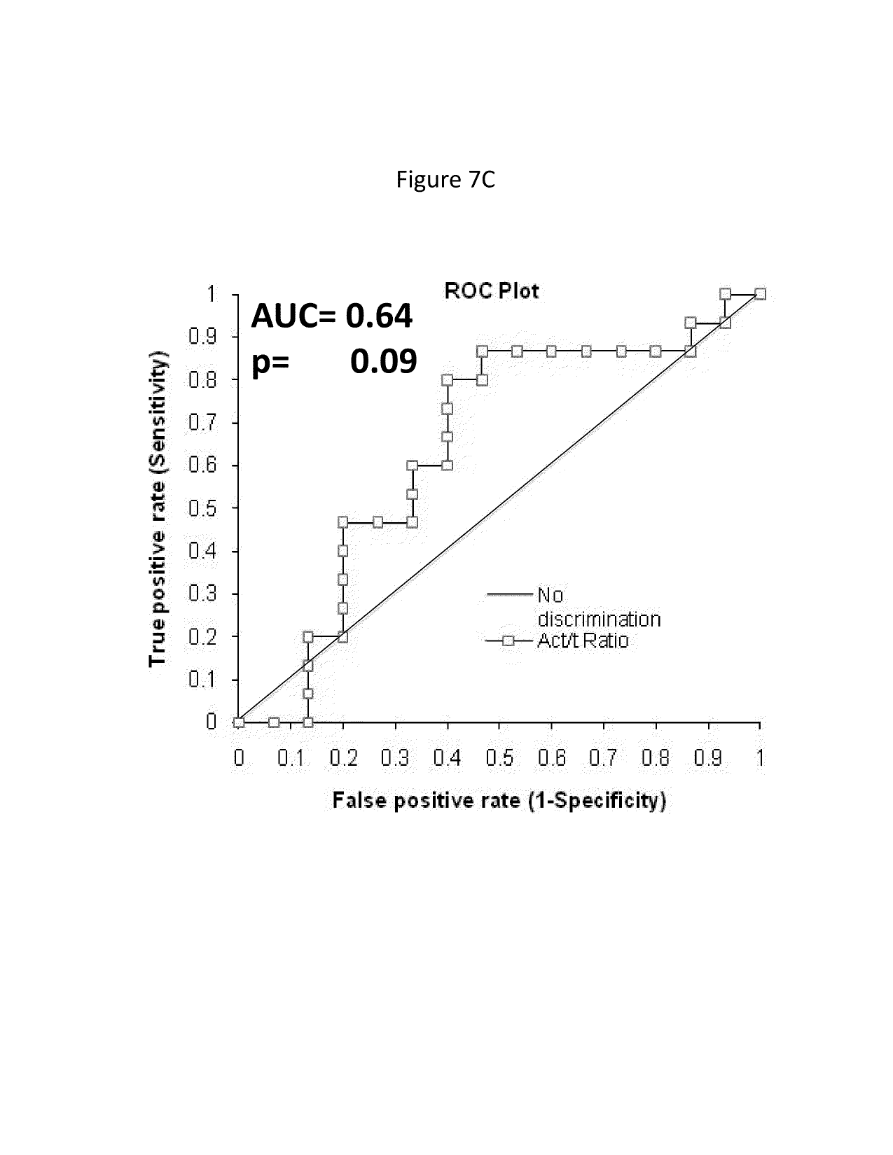

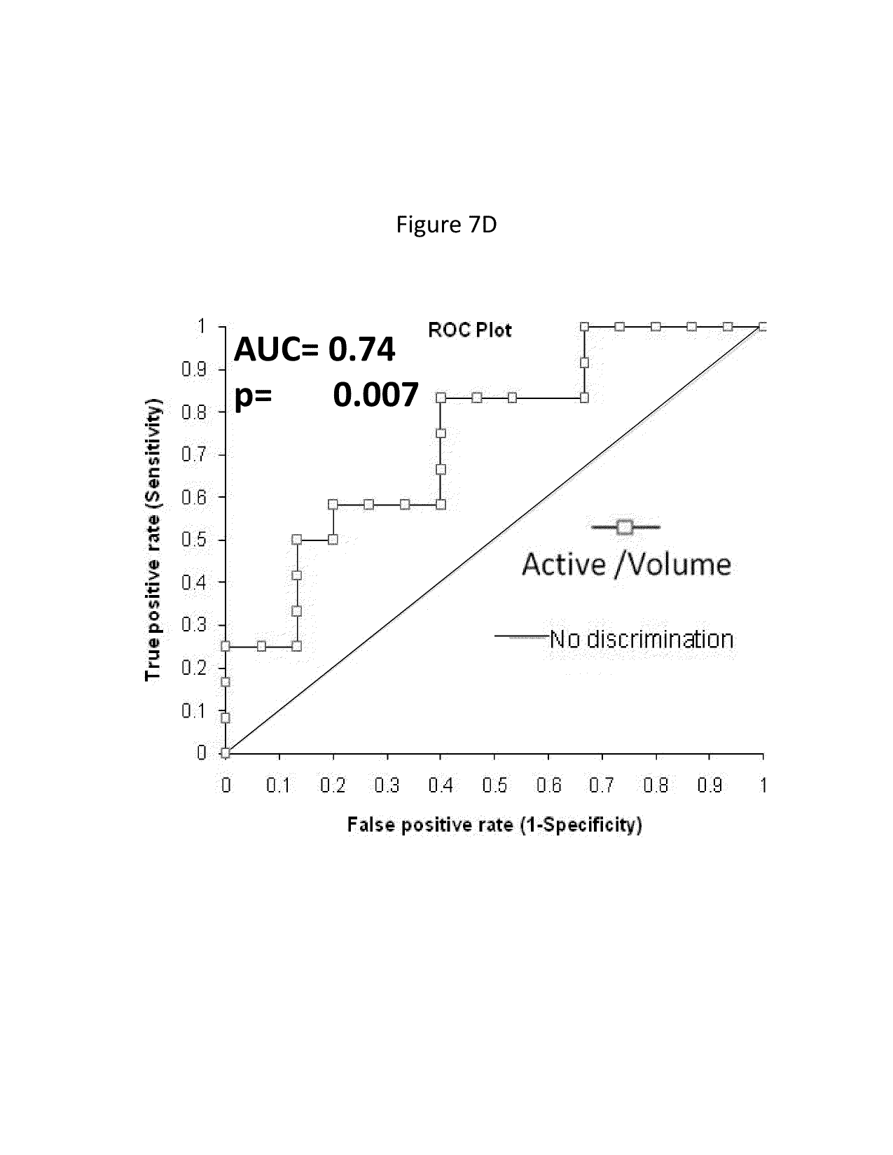

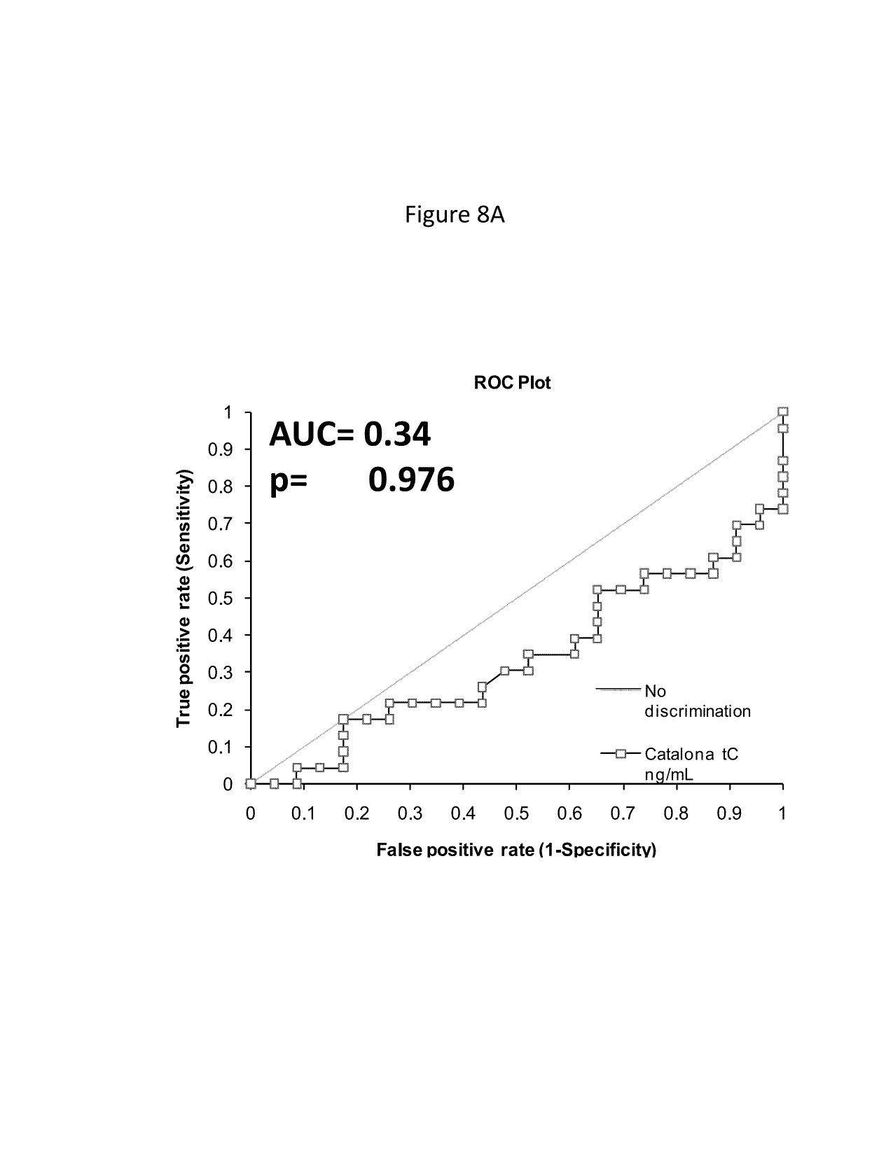

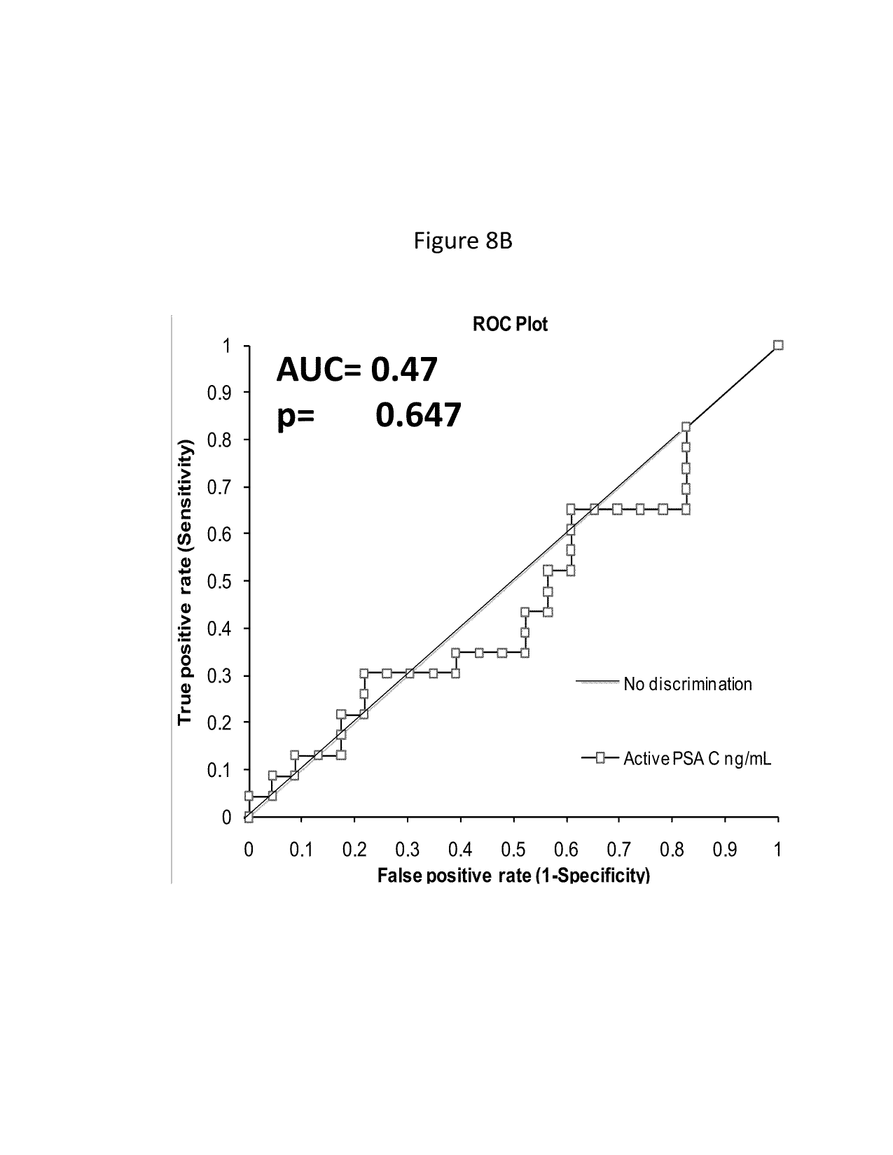

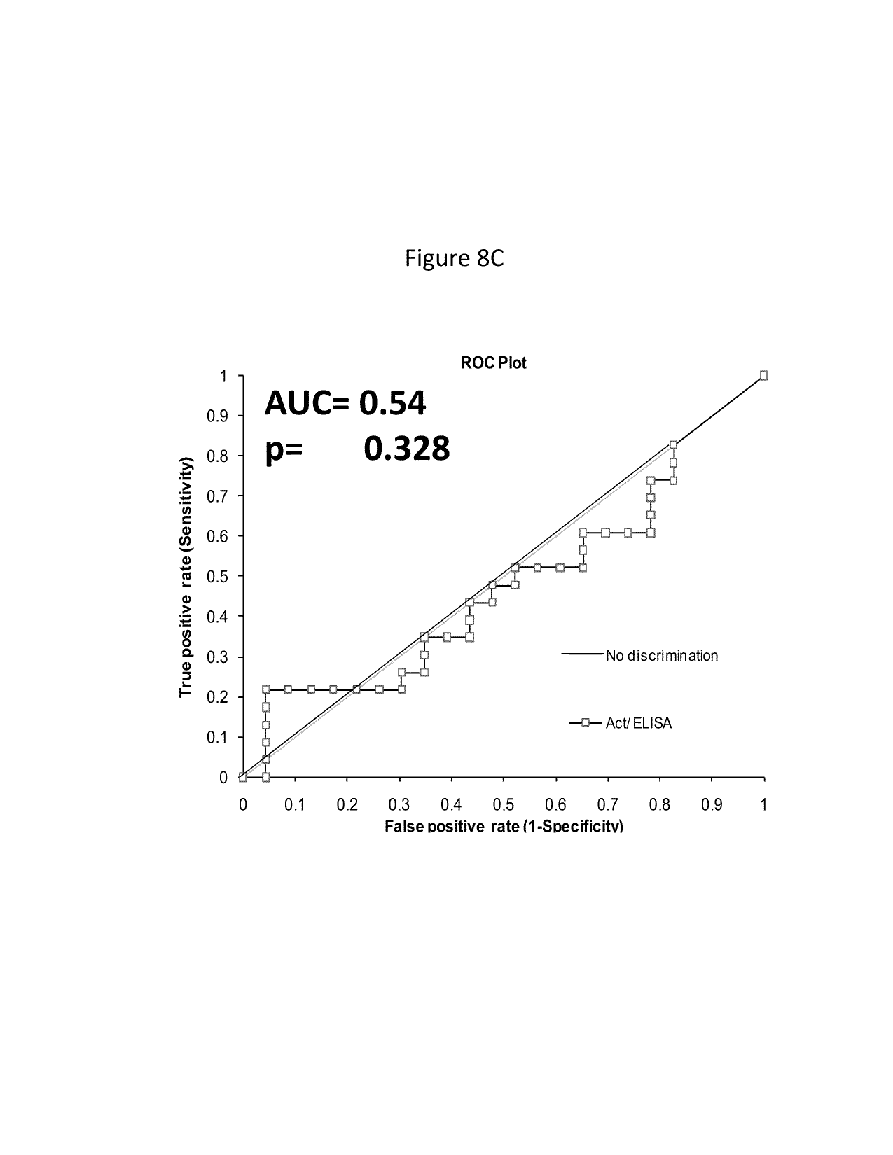

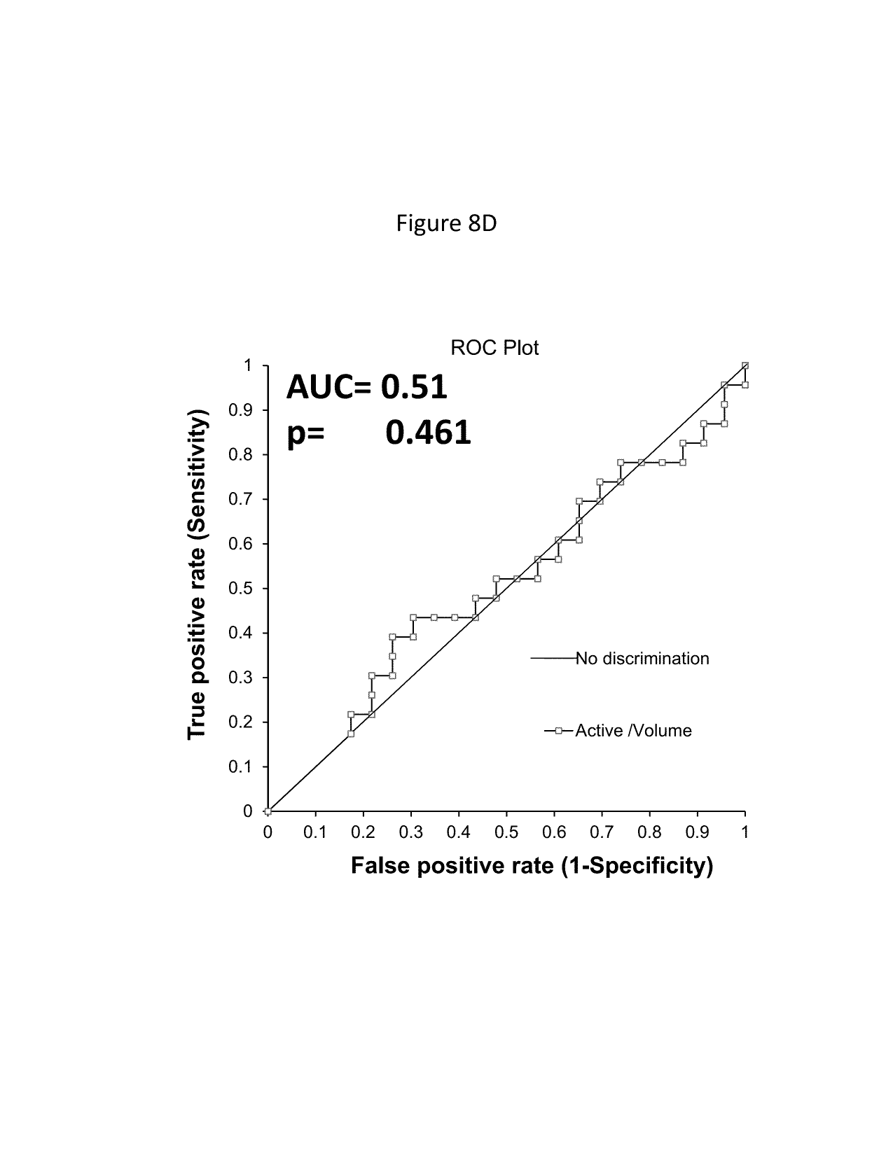

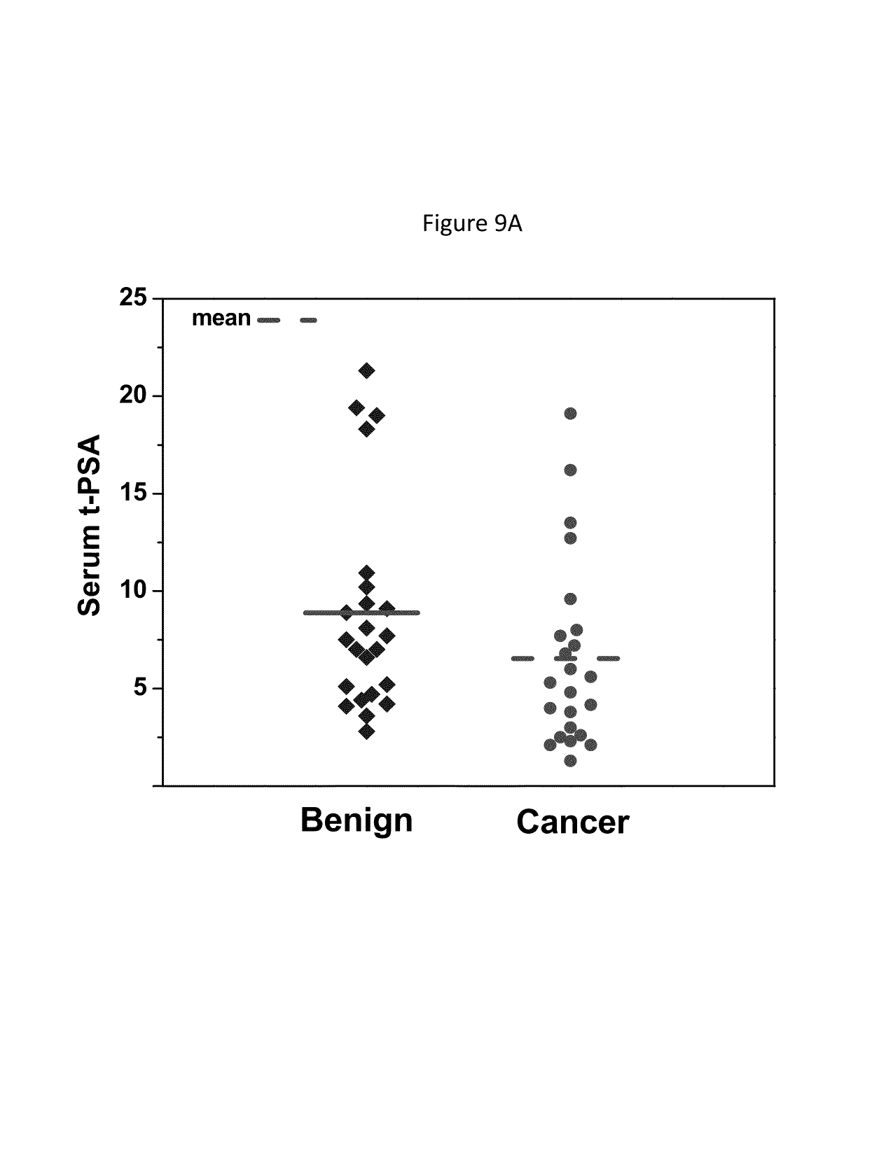

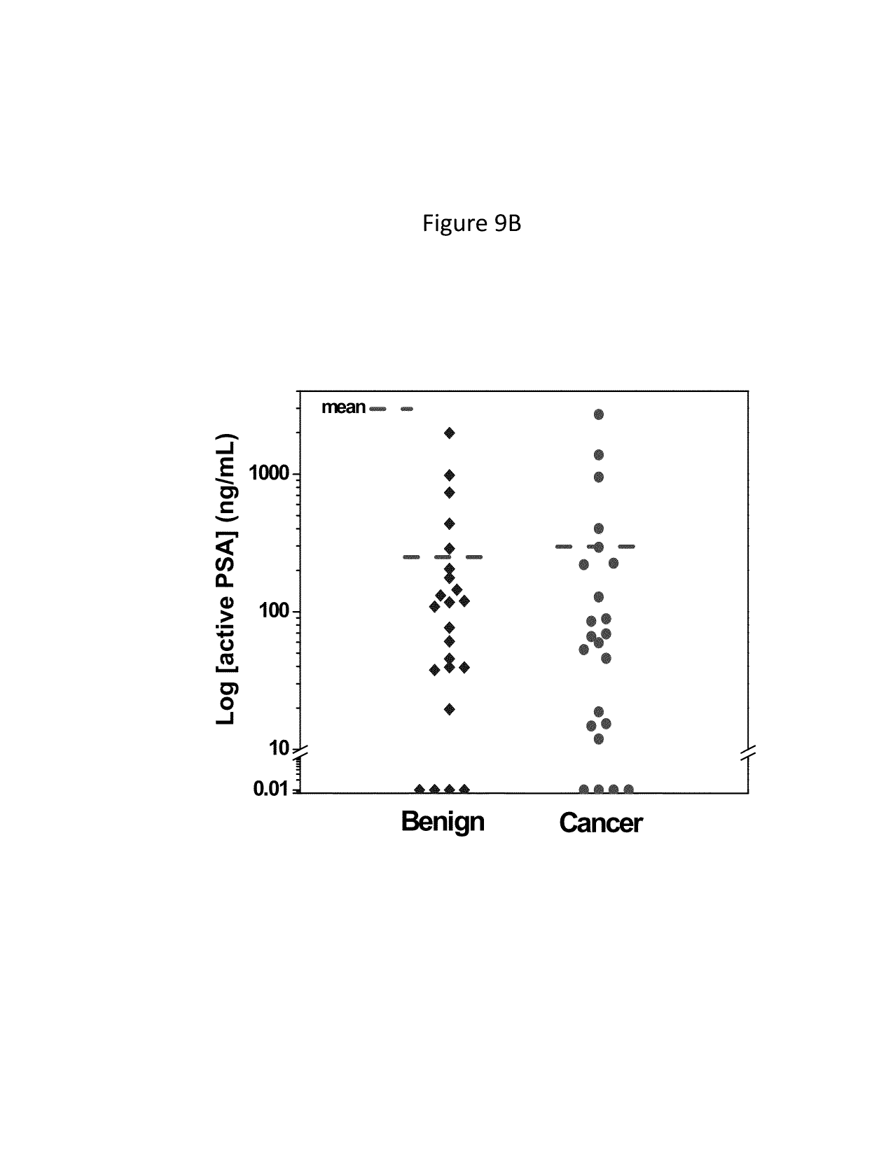

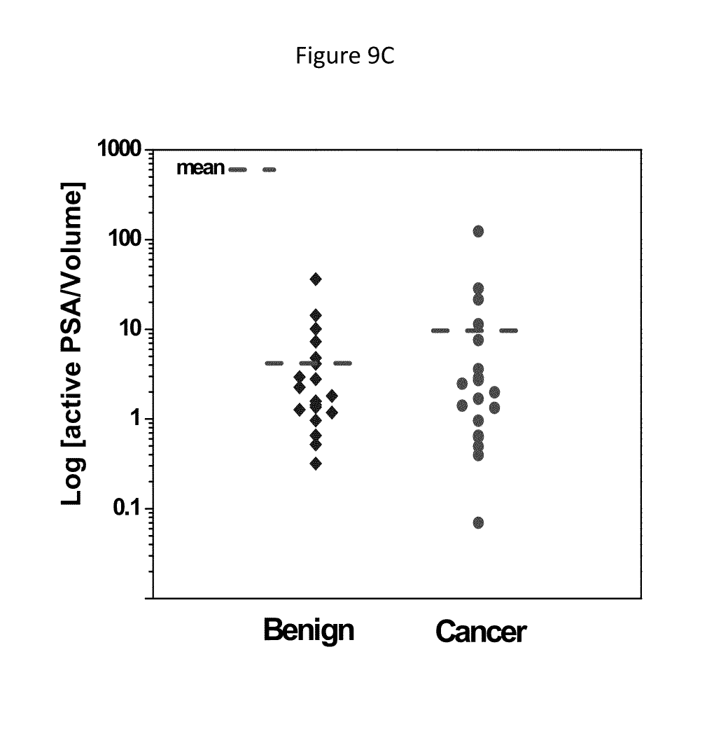

Once the activity data is normalized for the presence of total PSA and the prostate volume a better discrimination is once again shown. Again the same ROC curve analysis was carried out for all the relevant biomarkers discussed here and it is obvious that PSA proteolytic activity is a better biomarker than the serum t-PSA, as shown by the increasing area under the curve (AUC) values and the decreasing p values in the FIGS. 7A-7D.

### Example 5

Samples were and analyzed as described above in Example 2.A. To test whether the alternative substrates “HIC” and “HIV” that also show cleavage by PSA, similar to AMIDE peptide, could be hydrolyzed by other enzymes in the sample, particularly any esterases, control experiments were done. This cleavage event should not be detectable fluorometrically since a glutamine (Q) amino acid would remain attached to the fluorophore (AMC) preventing the generation of a fluorescent signal. Furthermore, PSA in the sample should not recognize this sequence (Q-AMC) and could therefore produce false negative results.

This was tested by running a urine sample (+peptide substrate; 0.4 mM) with and without a “sacrificial ester” (alanine methyl ester; 40 mM). The idea is that if there are esterases in the sample, adding a relatively high concentration of ester will prevent them from cleaving the peptide substrate and we should therefore see a higher turnover of substrate. The results from this single experiment indicate there is no difference between the sample run with ester and that run without ester. So the possible options are that a) esterases are not present this particular sample; b) If there are esterases present, they do not cleave the peptide substrate but do cleave the sacrificial ester and c) if there are esterases present, they do not cleave the sacrificial ester and do cleave the peptide substrate.

An additional factor to consider in this activity assay is the possibility of additional proteases in the urine (other than PSA, or additional isoforms of PSA) that could produce a positive signal. To demonstrate that PSA is the only protease acting on the peptide substrate we ran two samples with and without a monoclonal antibody (“mAb”, available from Dako, mAb 0750, clone ER-PR8) that was shown to exhibit anti-catalytic activity for PSA. For both samples, there was an observed reduction in activity, but not a complete loss of signal. The possibilities include a) a higher concentration of mAb is needed to completely shut down the PSA activity or b) there are other proteases in the sample that are active and cleaving the substrate which is currently being further studied.

Esterases do not interfere with the assay described in this specification because the cleavage location is at the P2 position on the synthetic peptide substrate (instead of the P1 position) meaning the resulting product (Hic-Q-Tag) is not a fluorescent species nor a substrate for PSA. Furthermore, since the synthetic peptide substrate is intentionally added in significant excess to the sample, any esterase depletion of the substrate would have negligible effect on the measurement of target PSA activity by lowing effective substrate concentration.

It is understood that the examples and embodiments described herein are for illustrative purposes only. Unless clearly excluded by the context, all embodiments disclosed for one aspect of the invention can be combined with embodiments disclosed for other aspects of the invention, in any suitable combination. It will be apparent to those skilled in the art that various modifications and variations can be made to the present invention without departing from the scope of the invention. Thus, it is intended that the present invention cover the modifications and variations of this invention provided they come within the scope of the appended claims and their equivalents. All publications, patents, and patent applications cited herein are hereby incorporated herein by reference for all purposes.

What is claimed is:

1. A method for determining the prognosis of prostate cancer in a subject comprising:
a) contacting a sample taken from a subject with a labeled prostate cancer specific peptide that is a substrate of prostate specific antigen (PSA), said sample selected from the group consisting of urine, semen, prostatic fluid and post prostatic massage urine;
b) determining the level of PSA proteolytic activity in said sample;
c) normalizing said level of proteolytic activity to (i) total PSA in said sample; (ii) total PSA in the serum of said subject; or (iii) prostate volume; and
d) utilizing said normalized proteolytic activity level to prognose prostate cancer in said subject.

2. A method for monitoring the progression of prostate cancer therapy in a subject comprising:
a) contacting a sample taken from a subject with a labeled prostate cancer specific peptide that is a substrate of prostate specific antigen (PSA), said sample selected from the group consisting of urine, semen, prostatic fluid and post prostatic massage urine;
b) determining the level of PSA proteolytic activity in said sample;
c) normalizing said level of proteolytic activity to (i) total PSA in said sample; (ii) total PSA in the serum of said subject; or (iii) prostate volume; and
d) utilizing said normalized proteolytic activity level to monitor prostate cancer therapy in said subject.

3. A method for determining the aggressiveness of prostate cancer in a subject diagnosed with prostate cancer, said method comprising:
a) contacting a sample taken from a subject with a labeled prostate cancer specific peptide that is a substrate of prostate specific antigen (PSA), said sample selected from the group consisting of urine, semen, prostatic fluid and post prostatic massage urine;
b) determining the level of PSA proteolytic activity in said sample;
c) normalizing said level of proteolytic activity to (i) total PSA in said sample; (ii) total PSA in the serum of said subject; or (iii) prostate volume; and
d) utilizing said normalized proteolytic activity level to determine the aggressiveness of said prostate cancer in said subject.

4. A method according to claim 1 wherein said labeled prostate cancer specific peptide is labeled HSSKLQ (SEQ ID NO: 1).

5. A method according to claim 2 wherein said labeled prostate cancer specific peptide is labeled HSSKLQ (SEQ ID NO: 1).

6. A method according to claim 3 wherein said labeled prostate cancer specific peptide is labeled HSSKLQ (SEQ ID NO: 1).

7. A method according to claim 1 wherein said labeled prostate cancer specific peptide is labeled HSSK-Hiv-Q (SEQ ID NO: 21).

8. A method according to claim 2 wherein said labeled prostate cancer specific peptide is labeled HSSK-Hiv-Q (SEQ ID NO: 21).

9. A method according to claim 3 wherein said labeled prostate cancer specific peptide is labeled HSSK-Hiv-Q (SEQ ID NO: 21).

10. A method according to claim 1 wherein said labeled prostate cancer specific peptide is labeled HSSK-Hic-Q (SEQ ID NO: 22).

11. A method according to claim 2 wherein said labeled prostate cancer specific peptide is labeled HSSK-Hic-Q (SEQ ID NO: 22).

12. A method according to claim 3 wherein said labeled prostate cancer specific peptide is labeled HSSK-Hic-Q (SEQ ID NO: 22).

13. A method according to claim 1 wherein said label is chromogenic, fluorogenic, or electrochemical.

14. A method according to claim 2 wherein said label is chromogenic, fluorogenic, or electrochemical.

15. A method according to claim 3 wherein said label is chromogenic, fluorogenic, or electrochemical.

16. A method according to claim 1 wherein said labeled prostate cancer specific peptide is fibronectin.

17. A method according to claim 2 wherein said labeled prostate cancer specific peptide is fibronectin.

18. A method according to claim 3 wherein said labeled prostate cancer specific peptide is fibronectin.

19. A method according to claim 1 further comprising a step of providing said sample prior to determining the level of proteolytic activity in said sample.

20. A method according to claim 2 further comprising a step of providing said sample prior to determining the level of proteolytic activity in said sample.

21. A method according to claim 1 wherein said prostate cancer specific peptide ranges from 0.2 mM to 0.4 mM.

22. A method according to claim 2 wherein said prostate cancer specific peptide ranges from 0.2 mM to 0.4 mM.

23. A method according to claim 3 wherein said prostate cancer specific peptide ranges from 0.2 mM to 0.4 mM.
